(19) United States
(12) Patent Application Publication
(10) Pub. No.: US 2022/0056531 A1
(43) Pub. Date: Feb. 24, 2022

(54) PREDICTION AND CHARACTERIZATION OF DLBCL CELL OF ORIGIN SUBTYPES

(71) Applicants: Genentech, Inc., South San Francisco, CA (US); Hoffmann-La Roche Inc., Little Falls, NJ (US); Foundation Medicine, Inc., Cambridge, MA (US)

(72) Inventors: Sally Elizabeth TRABUCCO, Watertown, MA (US); Christopher R. BOLEN, Martinez, CA (US); Mikkel Zahle OESTERGAARD, Zollikon (CH); Ethan Samuel SOKOL, Somerville, MA (US); Lee Alan ALBACKER, Arlington, MA (US)

(21) Appl. No.: 17/162,718

(22) Filed: Jan. 29, 2021

Related U.S. Application Data
(63) Continuation of application No. PCT/US2019/044497, filed on Jul. 31, 2019.
(60) Provisional application No. 62/713,434, filed on Aug. 1, 2018.

(51) Int. Cl. C12Q 1/6886; C12Q 1/6806; G16H 50/20; G16H 50/30

(52) U.S. Cl. CPC C12Q 1/6886; G16H 50/30; G16H 50/20; C12Q 1/6806

(57) ABSTRACT

Provided are methods of determining cell of origin (COO) for diffuse large B Cell lymphoma (DLBCL) using DNA for the analysis. The methods include identification of DLBCL COO as activated B Cell (ABC) and germinal center B Cell (GCB).

### CROSS REFERENCE TO RELATED APPLICATION

This application is a continuation of International Application No. PCT/US2019/044497, filed 31 Jul. 2019, which claims the benefit of U.S. Provisional Application No. 62/713,434, filed 1 Aug. 2018, the contents of each of which are incorporated herein by reference in their entirety.

### BACKGROUND OF THE INVENTION

Diffuse large B Cell lymphoma (DLBCL) is the most common form of non-Hodgkin's lymphoma, accounting for more than 25,000 new cases per year in the United States (R., T. L., et al. 2016 US lymphoid malignancy statistics by World Health Organization subtypes. CA: A Cancer Journal for Clinicians 66, 443-459 (2016)). Prognosis for patients with DLBCL is fairly good, with five-year survival rates between 55-62% (R., T. L., et al. 2016 US lymphoid malignancy statistics by World Health Organization subtypes. CA: A Cancer Journal for Clinicians 66, 443-459 (2016)). However, relapsed patients and those who do not initially respond to standard of care continue to have limited options, primarily consisting of autologous stem cell transplants or clinical trials. Additionally, although 60-65% of patients fully respond to R-CHOP (Rituximab, cyclophosphamide, doxorubicin, vincristine, and prednisone) therapy, there is a significant difference in response rates according to the DLBCL cell of origin (COO).

DLBCL has two COO subtypes: Activated B Cell (ABC) and Germinal Center B cell (GCB). The ABC subtype has a poor prognosis compared with GCB, and COO can be predictive for response to some new therapeutic agents. Traditionally, COO subtype has been determined by microarray assessed expression (ABC, GCB, unclassified), immunohistochemistry (IHC)-based algorithms (GCB or non-GCB), and expression based assays such as the Nanostring research use only lymphoma subtyping (LST) assay (ABC, GCB, unclassified) from Nanostring Technologies (also referred to herein as the Nanostring assay or Nanostring). Unfortunately, microarray is not typically feasible and some reports have failed to show a prognostic difference between GCB and non-GCB by IHC-based algorithms. This has led some to adopt the Nanostring assay as the preferred method to assess COO, but in some cases the tumor content or RNA quality is not sufficient to perform this assay. COO subtypes have differing gene mutations, with GCB typically characterized by EZH2 alterations and IGH:BCL2 translocations, while ABC is dominated by NF-KB and BCR signaling alterations such as MYD88 and CD79B. Here we utilize mutational differences in COO subtypes to develop a COO DNA classification (COODC) model to predict COO from DNA-based features on a clinically utilized platform.

COO origin subtyping was originally developed using RNA expression microarrays (Alizadeh, A. A., et al. Distinct types of diffuse large B Cell lymphoma identified by gene expression profiling. Nature 403, 503 (2000)) and is a necessary component of DLBCL clinical care per the 2016 updated WHO lymphoid neoplasm classifications (Swerdlow, S. H., et al. The 2016 revision of the World Health Organization classification of lymphoid neoplasms. Blood 127, 2375-2390 (2016)). RNA-based classifiers commonly split DLBCL into the activated B Cell (ABC) and germinal center (GC) B Cell (GCB) subtypes. DLBCL that does not fall into either of these categories are called unclassified. These subtypes are both prognostic and predictive. Among patients treated with modern anti-CD20 therapies, the ABC subtype has a worse prognosis, with a three-year progression free survival of 59% compared with 75% for GCB patients (Vitolo, U., et al. Obinutuzumab or Rituximab Plus Cyclophosphamide, Doxorubicin, Vincristine, and Prednisone in Previously Untreated Diffuse Large B Cell Lymphoma. Journal of Clinical Oncology 35, 3529-3537 (2017)). However, targeted therapeutics, including the BCR signaling inhibitor ibrutinib, have recently been developed that target pathways commonly altered in ABC. Ibrutinib has shown significant clinical benefit among patients with mutations in BCR signaling genes, which are common in the ABC subset (Wilson, W. H., et al. Targeting B cell receptor signaling with ibrutinib in diffuse large B cell lymphoma. Nature Medicine 21, 922 (2015)). It is clear that accurate assessment of COO subtype is critical for the appropriate clinical management of DLBCL.

In most clinical settings it is not feasible to perform microarray analyses for each patient. This has led to the development and adoption of immunohistochemistry (IHC) based algorithms to approximate COO. The most widely used of these is the Hans' algorithm (Hans, C. P., et al. Confirmation of the molecular classification of diffuse large B Cell lymphoma by immunohistochemistry using a tissue microarray. Blood 103, 275-282 (2003)), which is able to classify GCB and non-GC DLBCL, the latter of which includes both ABC and unclassified subtypes. Unfortunately, these algorithms do not reliably recapitulate the original prognostic findings, and in some cases IHC based algorithms show no significant survival difference between GCB and non-GCB types (Gribben, R. C., et al. Poor Concordance among Nine Immunohistochemistry Classifiers of Cell-of-Origin for Diffuse Large B Cell Lymphoma: Implications for Therapeutic Strategies. (2013); Meyer, P. N., et al. Immunohistochemical Methods for Predicting Cell of Origin and Survival in Patients With Diffuse Large B Cell Lymphoma Treated With Rituximab. Journal of Clinical Oncology 29, 200-207 (2011)).

Given the need for reliable, clinically useful methods to determine COO for DLBCL, recent efforts have focused on simpler, more streamlined methods, including the Nanostring assay (Scott, D. W., et al. Determining cell-of-origin subtypes of diffuse large B Cell lymphoma using gene expression in formalin-fixed paraffin-embedded tissue. Blood 123, 1214-1217 (2014)). This method uses RNA expression from 20 genes to determine COO with 98% concordance to microarray and 8.8% unclassified calls compared with 14.7% by the gold standard microarray. While this holds great promise for clinical utility, it requires RNA and samples with at least 60% tumor purity (i.e., tumor content) to achieve this accuracy (Scott, D. W., et al. Determining cell-of-origin subtypes of diffuse large B Cell lymphoma using gene expression in formalin-fixed paraffin-embedded tissue. Blood 123, 1214-1217 (2014)), which is not routinely accessible.

COO subtypes have differing gene mutations, with GCB typically characterized by EZH2 alterations and IGH:BCL2 translocations, while ABC is dominated by NF-KB and BCR signaling alterations such as MYD88 and CD79B. Chapuy and colleagues recently showed distinct mutational signatures in DLBCL samples, including a canonical activation-induced cytidine deaminase (AID) signature (Chapuy, B., et al. Molecular subtypes of diffuse large B cell lymphoma are associated with distinct pathogenic mechanisms and outcomes. Nature Medicine 24, 679-690 (2018)). Although previous reports do not identify mutational signature differences between subtypes, it is hypothesized that there are differences in mutational profiles of GCB and ABC tumors and these are most likely the result of distinct histories of precursor cells. GCB DLBCLs are thought to arise from germinal center (GC) B Cells, while ABC subtype is thought to arise from post-GC B Cells. During the antibody maturation process, GC B Cells are subject to class switch recombination (CSR) and somatic hypermutation (SHM) induced by AID protein that induces double strand breaks (DSB) and single base mutations. Recent studies have shown certain genes in DLBCL tumors have evidence of frequent AID induced alterations (Chapuy, B., et al. Molecular subtypes of diffuse large B cell lymphoma are associated with distinct pathogenic mechanisms and outcomes. Nature Medicine 24, 679-690 (2018); Schmitz, R., et al. Genetics and Pathogenesis of Diffuse Large B Cell Lymphoma. New England Journal of Medicine 378, 1396-1407 (2018)) and certain DLBCL-related genes, such as PIM1, are thought to be hotspots for off-target AID activity.

In order to solve the problem of finding a reliable diagnostic for a DLBCL COO that does not require RNA and does not require samples with at least 60% tumor purity, a DNA-only classifier using Foundation Medicine's FoundationOne®Heme platform was invented and is disclosed herein. It is shown that this assay maintains 89% (e.g., approximately 90%) concordance to Nanostring, and requires samples having only 20% tumor content (e.g., approximately 20% tumor content). Furthermore, it is shown that the COO classifications from this assay reliably recapitulate the prognostic differences observed in RNA-based classifications like Nanostring.

### BRIEF SUMMARY OF THE INVENTION

A DNA-based method to determine COO for DLBCL was invented and is disclosed herein. In certain embodiments, this assay maintains 89% concordance to Nanostring. In certain embodiments, this assay requires samples having 20% tumor content as compared to the 60% required for Nanostring.

In some embodiments, provided herein is a method of determining the cell of origin (COO) of diffuse large B Cell lymphoma (DLBCL) using a cell of origin DNA classification (COODC). In certain embodiments according to (or as applied to) any of the embodiments above, the COODC comprises: (a) acquiring, e.g., collecting, a sample, e.g., a clinical sample, from a patient diagnosed with DLBCL, (b) performing DNA sequencing on the sample, e.g., clinical sample, and (c) applying a pre-defined COODC classifier to a list of genomic features (e.g., one or more features described in Table 1) to calculate a predictor score. In certain embodiments according to (or as applied to) any of the embodiments above, the DNA sequencing is performed using the DNA component of the FoundationOne®Heme platform. In certain embodiments according to (or as applied to) any of the embodiments above, the DNA sequencing comprises targeted DNA-sequencing of approximately 300-500 genes. In certain embodiments according to (or as applied to) any of the embodiments above, the detection of alterations in the BCL2, EZH2, and TNSFRSF14 genes using the COODC predicts the COO of the DLBCL is a germinal center B Cell (GCB). In certain embodiments according to (or as applied to) any of the embodiments above, alterations in the BCL2, EZH2, and TNSFRSF14 genes are detected. In certain embodiments according to (or as applied to) any of the embodiments above, the detection of alterations in the chromosome 3q copy number, MYD88, and CD79B genes using the COODC predicts the COO of the DLBCL is an activated B Cell (ABC). In certain embodiments according to (or as applied to) any of the embodiments above, alterations in the chromosome 3q copy number, MYD88, and CD79B genes are detected. In certain embodiments according to (or as applied to) any of the embodiments above, the detection of alterations in the NOTCH1, NOTCH2, and BCL6 genes using the COODC predicts the COO of the DLBCL is an activated B Cell (ABC). In certain embodiments according to (or as applied to) any of the embodiments above, alterations in the NOTCH1, NOTCH2, and BCL6 genes are detected. In certain embodiments according to (or as applied to) any of the embodiments above, the detection of IGH:BCL2 rearrangements, CREBBP alterations, and TNFRSF14 alterations predicts the COO of the DLBCL is a GCB. In certain embodiments according to (or as applied to) any of the embodiments above, IGH:BCL2 rearrangements, CREBBP alterations, and TNFRSF14 alterations are detected. In certain embodiments according to (or as applied to) any of the embodiments above, the detection of CDKN2A/B deletions, CD79B alterations at amino acid 196, and MYD88 alterations at amino acid 265 predicts the COO of the DLBCL is an ABC. In certain embodiments according to (or as applied to) any of the embodiments above, a CDKN2A/B deletion, a CD79B alteration at amino acid 196, and an MYD88 alteration at amino acid 265 are detected. In certain embodiments according to (or as applied to) any of the embodiments above, a COSMIC signature 3 predicts the COO of the DLBCL is GCB. In certain embodiments according to (or as applied to) any of the embodiments above, a COSMIC signature 3 is detected. In certain embodiments according to (or as applied to) any of the embodiments above, the method further comprises the step of (d) classifying a patient having a predictor score below a low cutoff (e.g., a low pre-defined cutoff) as GCB, and a patient having a predictor score above a high cutoff (e.g., a high pre-defined cutoff) as ABC. In certain embodiments according to (or as applied to) any of the embodiments above, the method further comprises the step of (e) classifying a patient having a predictor score above or equal to the low cutoff (e.g., the low pre-defined cutoff) and below the high cutoff (e.g., the high pre-defined cutoff) as unclassified. In certain embodiments according to (or as applied to) any of the embodiments above, the COODC was developed using the methods described in Examples 3-9. In certain embodiments according to (or as applied to) any of the embodiments above, no RNA analysis is performed. In certain embodiments according to (or as applied to) any of the embodiments above, RNA analysis does not contribute substantially to the determination of the COO. In certain embodiments according to (or as applied to) any of the embodiments above, no immunohistochemistry (IHC) analysis is performed. In certain embodiments according to (or as applied to) any of the embodiments above, IHC analysis does not contribute substantially to the determination of the COO.

In some embodiments, provided herein is a method of determining whether the cell of origin (COO) of diffuse large B Cell lymphoma (DLBCL) is an activated B Cell (ABC) or a germinal center B Cell (GCB) using a cell of origin DNA classification (COODC) model. In certain embodiments according to (or as applied to) any of the embodiments above, the method comprises (a) acquiring, e.g., collecting, a sample, e.g., a clinical sample, of a patient diagnosed with DLBCL, (b) performing DNA sequencing on the sample, e.g., clinical sample, (c) applying the pre-defined COODC classifier to the list of genomic features (e.g., one or more features described in Table 1) to calculate a predictor score, and (d) classifying a patient having a predictor score below a low cutoff (e.g., a low pre-defined cutoff) as GCB, and a patient having a predictor score above or equal to a high cutoff (e.g., a high pre-defined cutoff) as ABC. In certain embodiments according to (or as applied to) any of the embodiments above, the method further comprises the step of (e) classifying a patient having a predictor score above or equal to the low cutoff (e.g., the low pre-defined cutoff) and below the high cutoff (e.g., the high pre-defined cutoff) as unclassified. In certain embodiments according to (or as applied to) any of the embodiments above, the DNA sequencing is performed using the DNA component of the FoundationOne®Heme platform. In certain embodiments according to (or as applied to) any of the embodiments above, the DNA sequencing comprises targeted DNA-sequencing of approximately 300-500 genes. In certain embodiments according to (or as applied to) any of the embodiments above, the detection of alterations in the BCL2, EZH2, and TNSFRSF14 genes using the COODC predicts the COO of the DLBCL is germinal center B Cell (GCB). In certain embodiments according to (or as applied to) any of the embodiments above, alterations in the BCL2, EZH2, and TNSFRSF14 genes are detected. In certain embodiments according to (or as applied to) any of the embodiments above, the detection of alterations in the chromosome 3q copy number, MYD88, and CD79B genes using the COODC predicts the COO of the DLBCL is an activated B Cell (ABC). In certain embodiments according to (or as applied to) any of the embodiments above, alterations in the chromosome 3q copy number, MYD88, and CD79B genes are detected. In certain embodiments according to (or as applied to) any of the embodiments above, the detection of alterations in the NOTCH1, NOTCH2, and BCL6 genes using the COODC predicts the COO of the DLBCL is an activated B Cell (ABC). In certain embodiments according to (or as applied to) any of the embodiments above, of alterations in the NOTCH1, NOTCH2, and BCL6 genes are detected. In certain embodiments according to (or as applied to) any of the embodiments above, the detection of IGH:BCL2 rearrangements, CREBBP alterations, and TNFRSF14 alterations predicts the COO of the DLBCL is GCB. In certain embodiments according to (or as applied to) any of the embodiments above, IGH:BCL2 rearrangements, CREBBP alterations, and TNFRSF14 alterations are detected. In certain embodiments according to (or as applied to) any of the embodiments above, the detection of CDKN2A/B deletions, CD79B alterations at amino acid 196, and MYD88 alterations at amino acid 265 predicts the COO of the DLBCL is ABC. In certain embodiments according to (or as applied to) any of the embodiments above, CDKN2A/B deletions, CD79B alterations at amino acid 196, and MYD88 alterations at amino acid 265 are detected. In certain embodiments according to (or as applied to) any of the embodiments above, a COSMIC signature 3 predicts the COO of the DLBCL is GCB. In certain embodiments according to (or as applied to) any of the embodiments above, a COSMIC signature 3 is detected. In certain embodiments according to (or as applied to) any of the embodiments above, the COODC was developed using the methods described in Examples 3-9. In certain embodiments according to (or as applied to) any of the embodiments above, no RNA analysis is performed. In certain embodiments according to (or as applied to) any of the embodiments above, RNA analysis does not contribute substantially to the determination of COO. In certain embodiments according to (or as applied to) any of the embodiments above, no immunohistochemistry (IHC) analysis is performed. In certain embodiments according to (or as applied to) any of the embodiments above, IHC does not contribute substantially to the determination of COO.

In some embodiments, provided herein is a method of designing a therapy for the treatment of diffuse large B Cell lymphoma (DLBCL) using a cell of origin DNA classification (COODC) model comprising: (a) acquiring, e.g., collecting, a sample, e.g., a clinical sample, of a patient diagnosed with DLBCL, (b) performing DNA sequencing on the sample, e.g., clinical sample, (c) applying the pre-defined COODC classifier to the list of genomic features (e.g., one or more features described in Table 1) to calculate a predictor score, and (d) classifying a patient having a predictor score below a low cutoff (e.g., a low pre-defined cutoff) as GCB, and a patient having a predictor score above or equal to a high cutoff (e.g., a high pre-defined cutoff) as ABC, wherein the patient is administered a therapy recommended for their COO. In certain embodiments according to (or as applied to) any of the embodiments above, the method further comprises the step of (e) classifying a patient having a predictor score above or equal to the low cutoff (e.g., the low pre-defined cutoff) and below the high cutoff (e.g., the high pre-defined cutoff) as unclassified. In certain embodiments according to (or as applied to) any of the embodiments above, the GCB COO patient is administered a therapy that is effective for GCB COO. In certain embodiments according to (or as applied to) any of the embodiments above, the ABC COO patient is administered a therapy that is effective for ABC COO.

In some embodiments, provided herein is a method of predicting response to therapy for the treatment of diffuse large B Cell lymphoma (DLBCL) using a cell of origin DNA classification (COODC) model comprising (a) acquiring, e.g., collecting, a sample, e.g., a clinical sample, of a patient diagnosed with DLBCL, (b) performing DNA sequencing on the sample, e.g., clinical sample, (c) applying the pre-defined COODC classifier to the list of genomic features (e.g., one or more features described in Table 1) to calculate a predictor score, and (d) classifying a patient having a predictor score below a low cutoff (e.g., a low pre-defined cutoff) as GCB, and a patient having a predictor score above or equal to a high cutoff (e.g., a high pre-defined cutoff) as ABC, wherein the response to a therapy for the treatment of DLBCL is dependent on the COO. In certain embodiments according to (or as applied to) any of the embodiments above, the method further comprises the step of (e) classifying a patient having a predictor score above or equal to the low cutoff (e.g., the low pre-defined cutoff) and below the high cutoff (e.g., the high pre-defined cutoff) as unclassified. In certain embodiments according to (or as applied to) any of the embodiments above, the ABC COO is predictive for response to therapies known to be effective on the ABC subtype. In certain embodiments according to (or as applied to) any of the embodiments above, the therapy comprises ibrutinib and/or lenalidomide. In certain embodiments according to (or as applied to) any of the embodiments above, the GCB COO is predictive for response to therapies known to be effective on the GCB subtype. In certain embodiments according to (or as applied to) any of the embodiments above, the therapy comprises ibrutinib and/or lenalidomide. In certain embodiments according to (or as applied to) any of the embodiments above, the cell of origin (COO) of diffuse large B Cell lymphoma (DLBCL) is determined using a DNA-based platform method wherein neither RNA (e.g., analysis of RNA) nor immunohistochemistry is a component of the method. In certain embodiments according to (or as applied to) any of the embodiments above, the DNA-based platform is the COODC described in any of the embodiments described herein.

In some embodiments, provided herein is a cell of origin DNA classification (COODC) diagnostic used to determine whether the cell of origin (COO) of diffuse large B Cell lymphoma (DLBCL) is an activated B Cell (ABC) or a germinal center B Cell (GCB). In certain embodiments according to (or as applied to) any of the embodiments above, the COODC is the COODC described in any of the embodiments described herein. In certain embodiments according to (or as applied to) any of the embodiments above, the COODC is a kit.

In some embodiments, provided herein is a method for using a cell of origin DNA classification (COODC) diagnostic to determine whether the cell of origin (COO) of diffuse large B Cell lymphoma (DLBCL) is an activated B Cell (ABC) or a germinal center B Cell (GCB) comprising: (a) acquiring, e.g., collecting, a sample, e.g., a clinical sample, of a patient diagnosed with DLBCL, (b) performing DNA sequencing on the sample, e.g., clinical sample, (c) applying a COODC classifier (e.g., a pre-defined COODC classifier) to the list of genomic features (e.g., one or more features described in Table 1) to calculate a predictor score, and (d) classifying a patient having a predictor score below a low cutoff (e.g., a low pre-defined cutoff) as GCB, and a patient having a predictor score above or equal to a high cutoff (e.g., a high pre-defined cutoff) as ABC. In certain embodiments according to (or as applied to) any of the embodiments above, the method further comprises the step of (e) classifying a patient having a predictor score above or equal to the low cutoff (e.g., the low pre-defined cutoff) and below the high cutoff (e.g., the high pre-defined cutoff) as unclassified. In certain embodiments according to (or as applied to) any of the embodiments above, the diagnostic is a kit. In certain embodiments according to (or as applied to) any of the embodiments above, the COO is determined to be ABC or GCB according to any of any of the embodiments above.

In certain embodiments according to (or as applied to) any of the embodiments above, the sample is a clinical sample. In certain embodiments according to (or as applied to) any of the embodiments above, the clinical sample is tumor biopsy, blood, bone marrow aspirate, or an extracted nucleic acid. In certain embodiments according to (or as applied to) any of the embodiments above, the tumor biopsy is prepared on a formalin-fixed paraffin-embedded (FFPE) slide. In certain embodiments according to (or as applied to) any of the embodiments above, the DNA sequencing comprises targeted DNA-sequencing of approximately 465 genes. In certain embodiments according to (or as applied to) any of the embodiments above, a plurality of samples (e.g., a plurality of clinical samples), e.g., from a plurality of patients, are acquired (e.g., collected).

In some embodiments, provided herein is a method of selecting, classifying, or treating a subject comprising acquiring or providing a value for cell of origin (COO) and in response to the value selecting, classifying, evaluating, or treating the subject. In certain embodiments according to (or as applied to) any of the embodiments above, the value is acquired from another entity, e.g., a laboratory. In certain embodiments according to (or as applied to) any of the embodiments above, the value is substantially the same as the value that would be provided by the method or diagnostic of any of the embodiments described herein. In certain embodiments according to (or as applied to) any of the embodiments above, the value is determined by any of the embodiments described herein. In certain embodiments according to (or as applied to) any of the embodiments above, the subject has DLBCL.

In certain embodiments according to (or as applied to) any of the embodiments above, the low cutoff (e.g., the low pre-defined cutoff) for classifying patents or samples as GCB is between 0.2-0.3, e.g., between 0.21-0.29, 0.22-0.28, 0.23-0.27, 0.24-0.26, 0.21-0.3, 0.22-0.3, 0.23-0.3, 0.24-0.3, 0.25-0.3, 0.26-0.3, 0.27-0.3, 0.28-0.3, 0.29-0.3, 0.2-0.29, 0.2-0.28, 0.2-0.27, 0.2-0.26, 0.2-0.25, 0.2-0.24, 0.2-0.23, 0.2-0.22, or 0.2-0.21, e.g., 0.200, 0.201, 0.202, 0.203, 0.204, 0.205, 0.206, 0.207, 0.208, 0.209, 0.210, 0.211, 0.212, 0.213, 0.214, 0.215, 0.216, 0.217, 0.218, 0.219, 0.220, 0.221, 0.222, 0.223, 0.224, 0.225, 0.226, 0.227, 0.228, 0.229, 0.230, 0.231, 0.232, 0.233, 0.234, 0.235, 0.236, 0.237, 0.238, 0.239, 0.240, 0.241, 0.242, 0.243, 0.244, 0.245, 0.246, 0.247, 0.248, 0.249, 0.250, 0.251, 0.252, 0.253, 0.254, 0.255, 0.256, 0.257, 0.258, 0.259, 0.260, 0.261, 0.262, 0.263, 0.264, 0.265, 0.266, 0.267, 0.268, 0.269, 0.270, 0.271, 0.272, 0.273, 0.274, 0.275, 0.276, 0.277, 0.278, 0.279, 0.280, 0.281, 0.282, 0.283, 0.284, 0.285, 0.286, 0.287, 0.288, 0.289, 0.290, 0.291, 0.292, 0.293, 0.294, 0.295, 0.296, 0.297, 0.298, 0.299 or 0.300. In certain embodiments according to (or as applied to) any of the embodiments above, the high cutoff (e.g., the high pre-defined cutoff) for classifying samples as ABC is between 0.4-0.6, e.g., 0.41-0.59, 0.42-0.58, 0.43-0.57, 0.44-0.56, 0.45-0.55, 0.46-0.54, 0.47-0.53, 0.48-0.52, 0.49-0.51, 0.4-0.59, 0.4-0.58, 0.4-0.57, 0.4-0.56, 0.4-0.55, 0.4-0.54, 0.4-0.53, 0.4-0.52, 0.4-0.51, 0.4-0.50, 0.4-0.49, 0.4-0.48, 0.4-0.47, 0.4-0.46, 0.4-0.45, 0.4-0.44, 0.4-0.42, 0.4-0.41, 0.41-0.6, 0.42-0.6, 0.43-0.6, 0.44-0.6, 0.45-0.6, 0.46-0.6, 0.47-0.6, 0.48-0.6, 0.49-0.6, 0.5-0.6, 0.51-0.6, 0.52-0.6, 0.53-0.6, 0.54-0.6, 0.55-0.6, 0.56-0.6, 0.57-0.6, 0.58-0.6, or 0.59-0.6, e.g., 0.400, 0.401, 0.402, 0.403, 0.404, 0.405, 0.406, 0.407, 0.408, 0.409, 0.410, 0.411, 0.412, 0.413, 0.414, 0.415, 0.416, 0.417, 0.418, 0.419, 0.420, 0.421, 0.422, 0.423, 0.424, 0.425, 0.426, 0.427, 0.428, 0.429, 0.430, 0.431, 0.432, 0.433, 0.434, 0.435, 0.436, 0.437, 0.438, 0.439, 0.440, 0.441, 0.442, 0.443, 0.444, 0.445, 0.446, 0.447, 0.448, 0.449, 0.450, 0.451, 0.452, 0.453, 0.454, 0.455, 0.456, 0.457, 0.458, 0.459, 0.460, 0.461, 0.462, 0.463, 0.464, 0.465, 0.466, 0.467, 0.468, 0.469, 0.470, 0.471, 0.472, 0.473, 0.474, 0.475, 0.476, 0.477, 0.478, 0.479, 0.480, 0.481, 0.482, 0.483, 0.484, 0.485, 0.486, 0.487, 0.488, 0.489, 0.490, 0.491, 0.492, 0.493, 0.494, 0.495, 0.496, 0.497, 0.498, 0.499, 0.500, 0.501, 0.502, 0.503, 0.504, 0.505, 0.506, 0.507, 0.508, 0.509, 0.510, 0.511, 0.512, 0.513, 0.514, 0.515, 0.516, 0.517, 0.518, 0.519, 0.520, 0.521, 0.522, 0.523, 0.524, 0.525, 0.526, 0.527, 0.528, 0.529, 0.530, 0.531, 0.532, 0.533, 0.534, 0.535, 0.536, 0.537, 0.538, 0.539, 0.540, 0.541, 0.542, 0.543, 0.544, 0.545, 0.546, 0.547, 0.548, 0.549, 0.550, 0.551, 0.552, 0.553, 0.554, 0.555, 0.556, 0.557, 0.558, 0.559, 0.560, 0.561, 0.562, 0.563, 0.564, 0.565, 0.566, 0.567, 0.568, 0.569, 0.570, 0.571, 0.572, 0.573, 0.574, 0.575, 0.576, 0.577, 0.578, 0.579, 0.580, 0.581, 0.582, 0.583, 0.584, 0.585, 0.586, 0.587, 0.588, 0.589, 0.590, 0.591, 0.592, 0.593, 0.594, 0.595, 0.596, 0.597, 0.598, 0.599 or 0.600.

In certain embodiments according to (or as applied to) any of the embodiments above, if the predictor score is less than 0.3, e.g., less than 0.299, 0.298, 0.297, 0.296, 0.295, 0.294, 0.293, 0.292, 0.291, 0.29, 0.289, 0.288, 0.287, 0.286, 0.285, 0.284, 0.283, 0.282, 0.281, 0.28, 0.279, 0.278, 0.277, 0.276, 0.275, 0.274, 0.273, 0.272, 0.271, 0.27, 0.269, 0.268, 0.267, 0.266, 0.265, 0.264, 0.263, 0.262, 0.261, 0.26, 0.259, 0.258, 0.257, 0.256, 0.255, 0.254, 0.253, 0.252, 0.251, 0.25, 0.249, 0.248, 0.247, 0.246, 0.245, 0.244, 0.243, 0.242, 0.241, 0.24, 0.239, 0.238, 0.237, 0.236, 0.235, 0.234, 0.233, 0.232, 0.231, 0.23, 0.229, 0.228, 0.227, 0.226, 0.225, 0.224, 0.223, 0.222, 0.221, 0.22, 0.219, 0.218, 0.217, 0.216, 0.215, 0.214, 0.213, 0.212, 0.211, 0.21, 0.209, 0.208, 0.207, 0.206, 0.205, 0.204, 0.203, 0.202, 0.201, 0.200, 0.19, 0.18, 0.17, 0.16, 0.15, 0.14, 0.13, 0.12, 0.11, 0.1, 0.09, 0.08, 0.07, 0.06, 0.05, 0.04, 0.03, 0.02, or 0.01, the patient or sample is classified as GCB. In certain embodiments according to (or as applied to) any of the embodiments above, if the predictor score is greater than or equal to 0.4, e.g., greater than or equal to 0.401, 0.402, 0.403, 0.404, 0.405, 0.406, 0.407, 0.408, 0.409, 0.410, 0.411, 0.412, 0.413, 0.414, 0.415, 0.416, 0.417, 0.418, 0.419, 0.420, 0.421, 0.422, 0.423, 0.424, 0.425, 0.426, 0.427, 0.428, 0.429, 0.430, 0.431, 0.432, 0.433, 0.434, 0.435, 0.436, 0.437, 0.438, 0.439, 0.440, 0.441, 0.442, 0.443, 0.444, 0.445, 0.446, 0.447, 0.448, 0.449, 0.450, 0.451, 0.452, 0.453, 0.454, 0.455, 0.456, 0.457, 0.458, 0.459, 0.460, 0.461, 0.462, 0.463, 0.464, 0.465, 0.466, 0.467, 0.468, 0.469, 0.470, 0.471, 0.472, 0.473, 0.474, 0.475, 0.476, 0.477, 0.478, 0.479, 0.480, 0.481, 0.482, 0.483, 0.484, 0.485, 0.486, 0.487, 0.488, 0.489, 0.490, 0.491, 0.492, 0.493, 0.494, 0.495, 0.496, 0.497, 0.498, 0.499, 0.500, 0.501, 0.502, 0.503, 0.504, 0.505, 0.506, 0.507, 0.508, 0.509, 0.510, 0.511, 0.512, 0.513, 0.514, 0.515, 0.516, 0.517, 0.518, 0.519, 0.520, 0.521, 0.522, 0.523, 0.524, 0.525, 0.526, 0.527, 0.528, 0.529, 0.530, 0.531, 0.532, 0.533, 0.534, 0.535, 0.536, 0.537, 0.538, 0.539, 0.540, 0.541, 0.542, 0.543, 0.544, 0.545, 0.546, 0.547, 0.548, 0.549, 0.550, 0.551, 0.552, 0.553, 0.554, 0.555, 0.556, 0.557, 0.558, 0.559, 0.560, 0.561, 0.562, 0.563, 0.564, 0.565, 0.566, 0.567, 0.568, 0.569, 0.570, 0.571, 0.572, 0.573, 0.574, 0.575, 0.576, 0.577, 0.578, 0.579, 0.580, 0.581, 0.582, 0.583, 0.584, 0.585, 0.586, 0.587, 0.588, 0.589, 0.590, 0.591, 0.592, 0.593, 0.594, 0.595, 0.596, 0.597, 0.598, 0.599, 0.60, 0.61, 0.62, 0.63, 0.64, 0.66, 0.66, 0.67, 0.68, 0.69, 0.70, 0.71, 0.72, 0.73, 0.74, 0.77, 0.76, 0.77, 0.78, 0.79, 0.80, 0.81, 0.82, 0.83, 0.84, 0.88, 0.86, 0.87, 0.88, 0.89, 0.90, 0.90, 0.91, 0.92, 0.93, 0.94, 0.99, 0.96, 0.97, 0.98, or 0.99, the patient or sample is classified as ABC.

In certain embodiments according to (or as applied to) any of the embodiments above, if the predictor score is less than the high pre-defined cutoff, but greater than or equal to the low pre-defined cutoff, then the patient or sample is unclassified. In certain embodiments according to (or as applied to) any of the embodiments above, if the predictor score is greater than or equal to a low pre-defined cutoff described herein (e.g., between 0.2-0.3) and less than a high pre-defined cutoff described herein (e.g., between 0.4-0.6), e.g., greater than or equal to (a) any one of 0.299, 0.298, 0.297, 0.296, 0.295, 0.294, 0.293, 0.292, 0.291, 0.29, 0.289, 0.288, 0.287, 0.286, 0.285, 0.284, 0.283, 0.282, 0.281, 0.28, 0.279, 0.278, 0.277, 0.276, 0.275, 0.274, 0.273, 0.272, 0.271, 0.27, 0.269, 0.268, 0.267, 0.266, 0.265, 0.264, 0.263, 0.262, 0.261, 0.26, 0.259, 0.258, 0.257, 0.256, 0.255, 0.254, 0.253, 0.252, 0.251, 0.25, 0.249, 0.248, 0.247, 0.246, 0.245, 0.244, 0.243, 0.242, 0.241, 0.24, 0.239, 0.238, 0.237, 0.236, 0.235, 0.234, 0.233, 0.232, 0.231, 0.23, 0.229, 0.228, 0.227, 0.226, 0.225, 0.224, 0.223, 0.222, 0.221, 0.22, 0.219, 0.218, 0.217, 0.216, 0.215, 0.214, 0.213, 0.212, 0.211, 0.21, 0.209, 0.208, 0.207, 0.206, 0.205, 0.204, 0.203, 0.202, 0.201 or 0.200 and (b) less than any one of 0.401, 0.402, 0.403, 0.404, 0.405, 0.406, 0.407, 0.408, 0.409, 0.410, 0.411, 0.412, 0.413, 0.414, 0.415, 0.416, 0.417, 0.418, 0.419, 0.420, 0.421, 0.422, 0.423, 0.424, 0.425, 0.426, 0.427, 0.428, 0.429, 0.430, 0.431, 0.432, 0.433, 0.434, 0.435, 0.436, 0.437, 0.438, 0.439, 0.440, 0.441, 0.442, 0.443, 0.444, 0.445, 0.446, 0.447, 0.448, 0.449, 0.450, 0.451, 0.452, 0.453, 0.454, 0.455, 0.456, 0.457, 0.458, 0.459, 0.460, 0.461, 0.462, 0.463, 0.464, 0.465, 0.466, 0.467, 0.468, 0.469, 0.470, 0.471, 0.472, 0.473, 0.474, 0.475, 0.476, 0.477, 0.478, 0.479, 0.480, 0.481, 0.482, 0.483, 0.484, 0.485, 0.486, 0.487, 0.488, 0.489, 0.490, 0.491, 0.492, 0.493, 0.494, 0.495, 0.496, 0.497, 0.498, 0.499, 0.500, 0.501, 0.502, 0.503, 0.504, 0.505, 0.506, 0.507, 0.508, 0.509, 0.510, 0.511, 0.512, 0.513, 0.514, 0.515, 0.516, 0.517, 0.518, 0.519, 0.520, 0.521, 0.522, 0.523, 0.524, 0.525, 0.526, 0.527, 0.528, 0.529, 0.530, 0.531, 0.532, 0.533, 0.534, 0.535, 0.536, 0.537, 0.538, 0.539, 0.540, 0.541, 0.542, 0.543, 0.544, 0.545, 0.546, 0.547, 0.548, 0.549, 0.550, 0.551, 0.552, 0.553, 0.554, 0.555, 0.556, 0.557, 0.558, 0.559, 0.560, 0.561, 0.562, 0.563, 0.564, 0.565, 0.566, 0.567, 0.568, 0.569, 0.570, 0.571, 0.572, 0.573, 0.574, 0.575, 0.576, 0.577, 0.578, 0.579, 0.580, 0.581, 0.582, 0.583, 0.584, 0.585, 0.586, 0.587, 0.588, 0.589, 0.590, 0.591, 0.592, 0.593, 0.594, 0.595, 0.596, 0.597, 0.598, 0.599 or 0.60, e.g., if the predictor score is between 0.2-0.6, 0.22-0.58, 0.24-0.56, 0.26-0.54, 0.28-0.52, 0.3-0.5, 0.32-0.48, 0.34-0.46, 0.36-0.44, 0.38-0.42, 0.2-0.4, 0.22-0.38, 0.24-0.36, 0.26-0.34, 0.28-0.32, 0.3-0.6, 0.32-0.58, 0.34-0.56, 0.36-0.54, 0.38-0.52, 0.4-0.5, 0.42-0.48, or 0.44-0.46, e.g., between 0.3-0.4, 0.31-0.39, 0.32-0.38, 0.33-0.37, 0.34-0.36, 0.3-0.39, 0.3-0.38, 0.3-0.37, 0.3-0.36, 0.3-0.35, 0.3-0.34, 0.3-0.33, 0.3-0.32, 0.3-0.31, 0.31-0.4, 0.32-0.4, 0.33-0.4, 0.34-0.4, 0.35-0.4, 0.36-0.4, 0.37-0.4, 0.38-0.4, 0.39-0.4, e.g., 0.3, 0.301, 0.302, 0.303, 0.304, 0.305, 0.306, 0.307, 0.308, 0.309, 0.31, 0.311, 0.312, 0.313, 0.314, 0.315, 0.316, 0.317, 0.318, 0.319, 0.32, 0.321, 0.322, 0.323, 0.324, 0.325, 0.326, 0.327, 0.328, 0.329, 0.33, 0.331, 0.332, 0.333, 0.334, 0.335, 0.336, 0.337, 0.338, 0.339, 0.34, 0.341, 0.342, 0.343, 0.344, 0.345, 0.346, 0.347, 0.348, 0.349, 0.35, 0.351, 0.352, 0.353, 0.354, 0.355, 0.356, 0.357, 0.358, 0.359, 0.36, 0.361, 0.362, 0.363, 0.364, 0.365, 0.366, 0.367, 0.368, 0.369, 0.37, 0.371, 0.372, 0.373, 0.374, 0.375, 0.376, 0.377, 0.378, 0.379, 0.38, 0.381, 0.382, 0.383, 0.384, 0.385, 0.386, 0.387, 0.388, 0.389, 0.39, 0.391, 0.392, 0.393, 0.394, 0.395, 0.396, 0.397, 0.398, 0.399, or 0.4, the patient or sample is unclassified.

Without wishing to be bound by theory, it is believed that in some embodiments, the model described herein, which is described, e.g., by the features and weights, can be used to generate fitted predictor scores (e.g., probabilities) for the ABC subtype and these predictor scores (e.g., probabilities) can then be used to determine which subtype the patient or sample is based on pre-determined cutoffs. For example, the optimal cutoffs (e.g., the cutoffs described herein) for the continuous probability score can be determined by maximizing Youden's J statistic (the point with the highest total sensitivity and specificity) in the training set and then adding a 0.1 probability buffer on both sides of the initial cutoff to define an unclassified region, e.g., as described in Example 3 (e.g., FIGS. 1A and 1B). In certain embodiments, the cutoff, e.g., the low pre-defined cutoff described herein or the high pre-defined cutoff described herein, is determined as described in Example 3 (e.g., FIGS. 1A and 1B).

In some embodiments provided herein is a method for treating a subject having DLBCL comprising determining if the subject has an ABC or GCB COO, comprising i) acquiring, e.g., collecting, a sample, e.g., a clinical sample, and ii) performing a genotyping assay on the sample to determine if the subject has an ABC or GCB COO, and if the subject has DLBCL of ABC COO, then administering ibrutinib and/or lenalidomide, and if the subject has DLBCL of the GC COO, then administering ibrutinib and/or lenalidomide. In certain embodiments according to (or as applied to) any of the embodiments above, the COO is determined using a method or diagnostic of any of the embodiments described herein.

In some embodiments, provided herein is a computer system configured to perform any of the methods, diagnostics, kits or classifiers of any of the embodiments described herein.

### BRIEF DESCRIPTION OF THE DRAWINGS

FIG. 1A and FIG. 1B show receiver operating characteristics (ROC) curves for the COODC model. FIG. 1A shows ROC curves indicating sensitivity and specificity of the model for ABC and GCB for the GOYA training set and FIG. 1B shows ROC curves indicating sensitivity and specificity of the model for ABC and GCB for the GOYA held-out validation set.

FIGS. 2A-2F show concordance of COODC determined COO with Nanostring determined COO for GOYA samples. FIG. 2A shows pie graphs showing the overall breakdown of COO for all GOYA samples as determined by Nanostring or COODC. FIG. 2B shows pie graphs showing the breakdown of COODC COO calls within each Nanostring COO group. Survival curves indicating progression free survival for (FIG. 2C) COODC determined COO groups and (FIGS. 2D, 2E, 2F) COODC determined COO compared with Nanostring determined COO.

FIGS. 3A-3C show concordance of COODC determined COO with Nanostring determined COO for MAIN samples. FIG. 3A shows pie graphs showing the overall breakdown of COO for all MAIN samples as determined by Nanostring or COODC. FIG. 3B shows pie graphs showing the breakdown of COODC COO calls within each Nanostring COO group for MAIN. FIG. 3C shows a pie graph showing overall breakdown of COO for Foundation Medicine's deidentified genomic research database (“FM-clinical”) samples as determined by COODC.

FIG. 4A-4C show the enrichment of the COODC model features by COO. Enrichment is assessed using Nanostring assessed COO. FIG. 4A shows enrichment for all binary features with non-zero weights in the model. Enrichment in ABC is indicated by the darker grey dots (Log 2 Odds Ratio >0), enrichment in GCB is indicated by orange dots (Log 2 Odds Ratio <0). Dot sizes indicate the frequency of the feature in the enriched group. Labels indicate features with significant enrichment (p<0.05). FIG. 4B shows enrichment for continuous features with non-zero weights in the model. GCB is always on the left hand side of each panel, and ABC is on the right hand side. The horizontal lines in each box represents the median of the feature while the lower and upper bounds of each box represent 25th percentile and 75th percentile, respectively. The whiskers associated with each box extend to extreme values of no more than 1.5 times the inter-quartile range beyond the applicable box. Points beyond whiskers are considered outliers and plotted individually. *** p<0.001, ** p<0.01, * p<0.05, ns=not significant. FIG. 4C shows enrichment of COODC binary features in unclassified vs non-unclassified COO. Enrichment is assessed using Nanostring assessed COO. It shows enrichment for all binary features with non-zero weights in the model. Enrichment in unclassified is indicated by grey dots (Log 2 Odds Ratio >0), and enrichment in non-unclassified is indicated by purple dots (Log 2 Odds Ratio <0). Dot sizes indicate the frequency of the feature in the enriched groups. Labels indicate features with significant enrichment in unclassified samples (p<0.05). Features enriched in non-unclassified are not shown, but rather ABC vs. GCB features are indicated in FIG. 4A.

FIGS. 5A-5D show mutation signatures by COO. FIG. 5A-1-5A-3 shows plots of trinucleotide context for signature 3 (“BRCA” signature) (top) and signature 23 (bottom). FIG. 5A describes the positional arrangement between FIGS. 5A-1, 5A-2 and 5A-3. FIG. 5B shows frequency of mutational signatures by COODC type for GOYA samples in baited genomic regions as described in the methods. FIG. 5C shows frequency of mutational signatures by COODC type for FM-Clinical samples in baited genomic regions as described in the methods. Only samples with >=20 assessable mutations are included. If no dominant signature (i.e., no signature with >0.4 score) was detected, the signature is reported as “None”. FIG. 5D shows boxes (with whiskers) of all alterations assessed for trinucleotide context (left) and for TMB (right), with the median indicated by the horizontal line in each box. The lower and upper bounds of each box represent 25^{th} percentile and 75^{th} percentile, respectively. The whiskers associated with each box extend to extreme values of no more than 1.5 times the inter-quartile range beyond the box. Points beyond whiskers are considered outliers and plotted individually. *** p<0.001, ** p<0.01, * p<0.05, ns=not significant.

FIG. 6A and FIG. 6B show other genetically defined subsets of DLBCL in GOYA samples. FIG. 6A shows frequency of subsets approximating BN2, N1, MCD, and EZB groups identified by Schmitz and colleagues (Schmitz, R., et al. Genetics and Pathogenesis of Diffuse Large B Cell Lymphoma. New England Journal of Medicine 378, 1396-1407 (2018)). “Fit multiple” indicates samples that qualified for more than one genetically defined subset. Samples that were not qualified for any genetically defined subset were grouped based on the COODC subtype. FIG. 6B shows frequency of subsets approximating C1-05 groups identified by Chapuy and colleagues (Chapuy, B., et al. Molecular subtypes of diffuse large B cell lymphoma are associated with distinct pathogenic mechanisms and outcomes. Nature Medicine 24, 679-690 (2018)). “Fit multiple” indicates samples that qualified for more than one genetically defined subset. Samples that were not qualified for any genetically defined subset were grouped based on the COODC subtype.

FIG. 7A and FIG. 7B show other genetically defined subsets of DLBCL in FM-clinical samples. FIG. 7A shows frequency of subsets approximating BN2, N1, MCD, and EZB groups identified by Schmitz and colleagues (Schmitz, R., et al. Genetics and Pathogenesis of Diffuse Large B Cell Lymphoma. New England Journal of Medicine 378, 1396-1407 (2018)). “Fit multiple” indicates samples that qualified for more than one genetically defined subset. Samples that were not qualified for any genetically defined subset were grouped based on the COODC subtype. FIG. 7B shows frequency of subsets approximating C1-C5 groups identified by Chapuy and colleagues (Chapuy, B., et al. Molecular subtypes of diffuse large B cell lymphoma are associated with distinct pathogenic mechanisms and outcomes. Nature Medicine 24, 679-690 (2018)). “Fit multiple” indicates samples that qualified for more than one genetically defined subset. Samples that were not qualified for any genetically defined subset were grouped based on the COODC subtype.

### DETAILED DESCRIPTION OF THE INVENTION

Disclosed herein is a new, clinically relevant, method for determining the COO for DLBCL samples with approximately 20% tumor purity (i.e., tumor content) and without the need for RNA or matched normal tissue. This method is ˜90% concordant with the Nanostring assay compared to both a held-out validation set as well as an independent cohort. It was found that copy number alterations of chromosome 6p and 9p as well as the frequency of T>A and T>G transversions to be distinct between ABC and GCB subtypes. Finally, it was found that the mutational signatures underlying ABC are different from those found in GCB, suggesting differing roles of AID (activation-induced cytidine deaminase) in development of these two subtypes.

Current WHO guidelines require COO determination for newly diagnosed DLBCLs, but it is clinically challenging to obtain a standardized, rigorous determination for most samples (He, J., et al. Integrated genomic DNA/RNA profiling of hematologic malignancies in the clinical setting. Blood 127, 3004-3014 (2016)). IHC algorithms vary in both feasibility and utility of the results (Hans, C. P., et al. Confirmation of the molecular classification of diffuse large B Cell lymphoma by immunohistochemistry using a tissue microarray. Blood 103, 275-282 (2003); Gribben, R. C., et al. Poor Concordance among Nine Immunohistochemistry Classifiers of Cell-of-Origin for Diffuse Large B Cell Lymphoma: Implications for Therapeutic Strategies. (2013); Meyer, P. N., et al. Immunohistochemical Methods for Predicting Cell of Origin and Survival in Patients With Diffuse Large B Cell Lymphoma Treated With Rituximab. Journal of Clinical Oncology 29, 200-207 (2011)). The Nanostring assay has begun to emerge as a more clinically feasible alternative that evaluates the expression of a small subset of genes. However, the published validation for this assay, which is 98% concordant with the microarray assay, requires >60% tumor content as well as high quality RNA, which is often not available in the clinical setting (Scott, D. W., et al. Determining cell-of-origin subtypes of diffuse large B Cell lymphoma using gene expression in formalin-fixed paraffin-embedded tissue. Blood 123, 1214-1217 (2014)). Herein is described a new classifier that is ˜90% concordant with Nanostring, requires only 20% tumor content (e.g., approximately 20% tumor content), and does not require RNA, which is more susceptible to degradation or lower quality than DNA. Based on data from FM-clinical samples, an estimated 50% of DLBCL samples would not be eligible or results may be unreliable for expression-panel COO determination such as Nanostring due to either missing RNA or having <60% tumor purity. Therefore, this method as applied clinically will greatly increase the number of samples with a reliable COO determination.

It has been shown that more granular classification of DLBCL utilizing genomic alterations can provide more insight into prognosis (Chapuy, B., et al. Molecular subtypes of diffuse large B cell lymphoma are associated with distinct pathogenic mechanisms and outcomes. Nature Medicine 24, 679-690 (2018); Schmitz, R., et al. Genetics and Pathogenesis of Diffuse Large B Cell Lymphoma. New England Journal of Medicine 378, 1396-1407 (2018)). The mutations utilized in the COO prediction model disclosed herein shared significant overlap with the mutations highlighted by these papers. As discovered and disclosed herein, the alterations in BCL2, EZH2, and TNFRSF14 are all important predictors of the GCB subtype, consistent with the enrichment of GCB samples among the EZB cluster in Schmitz et al., or the C3 group DLBCLs in Chapuy et al. (see references above). Similarly, mutations in MYD88 and CD79B were highly predictive of an ABC subtype. Interestingly, it was discovered and disclosed herein that mutations in NOTCH1, NOTCH2, and BCL6 are all slightly predictive of ABC subtype, despite the mixed subtypes found in the BN2, N1 and C1 groups. However, these alterations were given less weight than other ABC-specific alterations, meaning that mutated samples would be more likely to fall into the “unclassified” subgroup.

It was also discovered and disclosed herein that many of the arm level copy number alterations in the COODC model are similar to the C2 group DLBCL in Chapuy et al. (see above), including 17p (both average copy number and fraction under loss of heterozygosity (LOH)), 6p (average copy number), and 5p (average copy number). In addition to these full arm level events, a number of partial arm level alterations correlate with full arm level alterations included in the COODC model such as 4q351 and 4q21.22 in the C2 group and 4q fraction under LOH in COODC. This suggests that many of the genomic groups identified by Schmitz and Chapuy overlap with the genomically defined COODC disclosed herein (see Schmitz et al., and Chapuy et al).

Approximate subsets similar to the BN2, N1, MCD, and EZB groups identified by Schmitz and C1-C5 groups identified by Chapuy and colleagues were used herein, illustrating that further subsetting into rough approximations of these populations should be feasible on a large scale clinical assay, which could provide more granular prognostic information. Additionally, provided herein are therapeutically relevant biomarkers, including BCR-dependence, TP53 wild type, EZH2 alterations, and BCL2 alterations. Given the high frequency of samples in the DLBCL datasets used for the inventions and discoveries disclosed herein with biomarkers associated with targeted therapies (85% in GOYA study samples and 81% in FM-clinical samples), it is clear that adding COO calling capabilities to next generation sequencing (NGS) provides both prognostic and therapeutically relevant actionability information for DLBCL patients.

It was also found, using the DLBCL datasets disclosed herein, that there are similarities in mutational signatures to those identified by Chapuy.

Two major mutational signatures in DLBCL samples were discovered: COSMIC signature 3, annotated as BRCA signature, and COSMIC signature 23. COSMIC signature 23, which was found primarily in the ABC subtype, has frequent alterations in the R[C]Y context, which suggests a canonical AID signature. Interestingly, COSMIC signature 3 is common among all DLBCL subtypes, found in 26% of GCBs, 16% of ABCs, and 25% of unclassified from COODC GOYA samples when a signature could be determined. Given that COSMIC signature 3 appears to capture the effects of DSB resolved by non-homologous end joining (NHEJ) when BRCA1/2 is absent, this signature is likely capturing the resolution of AID-induced DSB, which also uses NHEJ (Kotnis, A., Du, L., Liu, C., Popov, S. W. & Pan-Hammarström, Q. Non-homologous end joining in class switch recombination: the beginning of the end. in Philos Trans R Soc Lond B Biol Sci, Vol. 364 653-665 (2009)), and is essential for CSR.

Additionally, it was discovered that almost exclusively COSMIC signature 3 is found across all subtypes region of the IGH locus, suggesting a shared mutational process.

These findings fit in a model whereby GCBs originate from a cell that has experienced the mutational stress of AID mediated DSB, while ABCs originate from a cell that has undergone mutational stress related to both AID-mediated DSB and AID-mediated single base alterations. This fits with the current model in which GCBs originate from an earlier germinal center B Cell, while ABCs originate from a developmentally more advanced post or late germinal center cell. This is especially interesting given that there is only a small difference in tumor mutational burden (TMB) between ABC and GCB subtypes (median ABC 8.9, GCB 12.1, p=0.011), and that there is a slightly lower number of mutations assessable for mutational signature calling (including silent and non-coding alterations) in ABC than in GCB (median ABC 23.5, GCB 31, p=0.0019). This suggests that, while some ABC cells experience both AID-mediated DSB and AID-mediated single base alterations, and while GCB cells appear to mainly experience AID-mediated DSB, the mutational processes that lead to transformation result in similar overall mutational burden.

In certain embodiments according to (or as applied to) any of the embodiments above, “sample” refers to a biological sample obtained or derived from a source of interest, as described herein. In some embodiments, a source of interest comprises an organism, such as an animal or human. The source of the sample can be solid tissue as from a fresh, frozen and/or preserved organ, tissue sample, biopsy, resection, smear, or aspirate; blood or any blood constituents; bodily fluids such as cerebral spinal fluid, amniotic fluid, peritoneal fluid or interstitial fluid; or cells from any time in gestation or development of the subject. In some embodiments, the source of the sample is blood or blood constituents.

In some embodiments, the sample is or comprises biological tissue or fluid. The sample can contain compounds that are not naturally intermixed with the tissue in nature such as preservatives, anticoagulants, buffers, fixatives, nutrients, antibiotics or the like. In one embodiment, the sample is preserved as a frozen sample or as formaldehyde- or paraformaldehyde-fixed paraffin-embedded (FFPE) tissue preparation. For example, the sample can be embedded in a matrix, e.g., an FFPE block or a frozen sample. In another embodiment, the sample is a blood or blood constituent sample. In yet another embodiment, the sample is a bone marrow aspirate sample. In another embodiment, the sample comprises cell-free DNA (cfDNA). Without wishing to be bound by theory, it is believed that in some embodiments, cfDNA is DNA from apoptosed or necrotic cells. Typically, cfDNA is bound by protein (e.g., histone) and protected by nucleases. CfDNA can be used as a biomarker for non-invasive prenatal testing (NIPT), organ transplant, cardiomyopathy, microbiome, and cancer. In another embodiment, the sample comprises circulating tumor DNA (ctDNA). Without wishing to be bound by theory, it is believed that in some embodiments, ctDNA is cfDNA with a genetic or epigenetic alteration (e.g., a somatic alteration or a methylation signature) that can discriminate it originating from a tumor cell versus a non-tumor cell. In another embodiment, the sample comprises circulating tumor cells (CTCs). Without wishing to be bound by theory, it is believed that in some embodiments, CTCs are cells shed from a primary or metastatic tumor into the circulation. In some embodiments, CTC apoptosis is a source of ctDNA in the blood/lymph.

In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or bronchoalveolar lavages; aspirates; scrapings; bone marrow specimens; tissue biopsy specimens; surgical specimens; feces, other body fluids, secretions, and/or excretions; and/or cells therefrom, etc.

In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by a method chosen from biopsy (e.g., fine needle aspiration or tissue biopsy), surgery, collection of body fluid (e.g., blood, lymph, or feces), etc. In some embodiments, as will be clear from context, the term “sample” refers to a preparation that is obtained by processing (e.g., by removing one or more components of and/or by adding one or more agents to) a primary sample, e.g., filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

In some embodiments, the sample is a cell associated with a tumor, e.g., a tumor cell or a tumor-infiltrating lymphocyte (TIL). In some embodiments, the sample includes one or more premalignant or malignant cells. In some embodiments, the sample is acquired from a hematologic malignancy (or premaligancy), e.g., a hematologic malignancy (or premaligancy) described herein, e.g., a diffuse large B-cell lymphoma (DLBCL). In other embodiments, the sample includes one or more circulating tumor cells (CTCs) (e.g., a CTC acquired from a blood sample). In some embodiments, the sample is a cell not associated with a tumor, e.g., a non-tumor cell or a peripheral blood lymphocyte.

### EXAMPLES

### Example 1: Samples

Clinical and genomic data was available from patients treated in the GOYA clinical trial (NCT01287741; R-CHOP vs G-CHOP) (Vitolo, U., et al. Obinutuzumab or Rituximab Plus Cyclophosphamide, Doxorubicin, Vincristine, and Prednisone in Previously Untreated Diffuse Large B Cell Lymphoma. Journal of Clinical Oncology 35, 3529-3537 (2017)) and MAIN clinical trial (NCT00486759; R-CHOP+/−Bevacizumab) (Seymour, J. F., et al. R-CHOP with or without bevacizumab in patients with previously untreated diffuse large B Cell lymphoma: final MAIN study outcomes. Haematologica 99, 1343-1349 (2014)). The protocols of the original trials were approved by local or national ethics committees according to the laws of each country, and the studies were undertaken in accordance with the Declaration of Helsinki. Activated B Cell (ABC)/germinal center B Cell (GCB)/Unclassified DLBCL prognostic subtypes were determined by the Nanostring assay. Median follow-up durations were 24 months and 29 months for MAIN and GOYA, respectively 30% of progression free survival (PFS) events in both cohorts). Routine clinical care samples (hereafter referred to as FM-clinical samples) were sequenced as part of routine clinical care and corresponding clinical data was not available.

### Example 2: DNA Sequencing

All samples were sequenced using the DNA component of the FoundationOne®Heme platform, which includes targeted DNA-sequencing of approximately 465 genes, as described in He, J., et al. Integrated genomic DNA/RNA profiling of hematologic malignancies in the clinical setting. Blood 127, 3004-3014 (2016). In addition to validated short variants, copy number alterations (high-level amplifications and deep deletions), rearrangements, tumor mutational burden (TMB), and microsatellite instability (MSI) status, research use only features of the platform were utilized including chromosomal arm level copy number and loss of heterozygosity (LOH) metrics.

### Example 3: Machine Learning

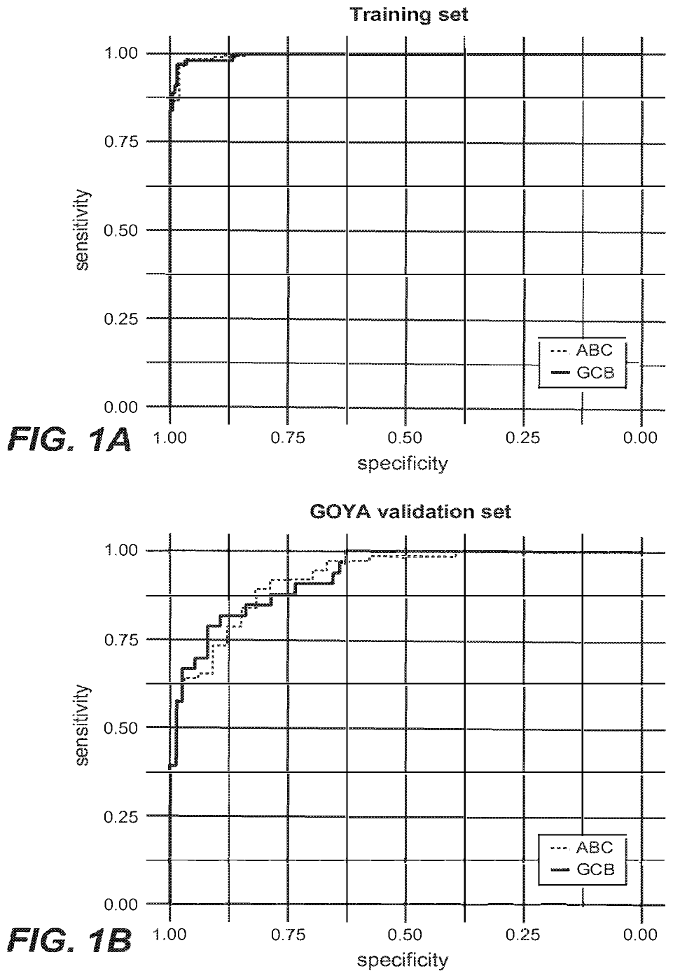

The COODC model was developed using a penalized Lasso regression using 25-fold internal cross validation implemented from the glmnet package (version 2.0-10) in R version 3.3.2 and using RStudio version 1.0.136. 482 GOYA samples with Nanostring data were split into a training set (70% of the samples) and a validation set (30% of the samples). The initial training set was further refined by removing Nanostring unclassified samples to focus the training to make ABC or GCB calls. 296 samples were used for the final training set, while 139 samples were used for the validation set. 592 features were used in the model. Continuous features were z-scored to maintain a consistent scale between continuous and binary features. The final COODC model included 74 non-zero features (Table 1). Per sample probabilities extracted from the model were used to determine the ideal cutoffs. ROC curves were generated (FIGS. 1A and 1B) and the “best” cutoff, which optimizes specificity and sensitivity, was chosen. 10% was then added on either side of the optimal cutoff to generate an unclassified zone. The probabilities were then extracted for the held-out validation set and 44 independent validation samples from the MAIN study. These two validation sets were used to determine the accuracy of the model.

TABLE 1
Features of the COODC model

| Features | Coefs |
|---|---|
| (Intercept) | 0.74622792 |
| T.A | 0.14266164 |
| T.G | 0.19169638 |
| chr2p_Num_Segments | 0.12336813 |
| chr3q_Average_CN | −0.5625825 |
| chr4q_Fraction_LOH | 0.00273763 |
| chr5q_Average_CN | 0.00758733 |
| chr6p_Average_CN | 0.00369147 |
| chr7q_Average_CN | 0.22046432 |
| chr8q_Average_CN | 0.00869661 |
| chr9p_Num_Segments | −0.0870849 |
| chr10p_Num_Segments | −0.0132862 |
| chr10p_Modal_CN | 0.04487006 |
| chr12p_Average_CN | 0.10812066 |
| chr12q_Average_CN | 0.33833335 |
| chr17p_Average_CN | 0.06825782 |
| chr17p_Fraction_LOH | −0.0668038 |
| chr17q_Fraction_LOH | 0.0772327 |
| chr18q_Average_CN | −0.0135074 |
| chr19q_Average_CN | −0.2309455 |
| chr19q_Modal_CN | −0.0159153 |
| ft_:_PIM1_\|_isKL_−_equals_−_KL | −0.2789378 |
| ft_:_NOTCH1_\|_variant_type_−_equals_−_SV_+_codon_−_equals_−_2514 | −0.7118127 |
| ft_:_BCOR_\|_variant_type_−_equals_−_SV_+_isKL_−_equals_−_KL | −0.4570838 |
| ft_:_TNFRSF14_\|_isKL_−_equals_−_KL | 0.30795551 |
| ft_:_BCL2_\|_isKL_−_equa1s_−_KL | 0.21222866 |
| ft_:_HIST1H2AM_LisKL_−_equals_−_KL | −1.1452282 |
| ft_:_CREBBP_\|_isKL_−_equals_−_KL | 0.39528297 |
| ft_:_GNA13_\|_variant_type_−_equals_−_SV_+_codon_−_equals_−_28 | 0.04776333 |
| ft_:_BCL10_\|_variant_type_−_equals_−_SV_+_isKL_−_equals_−_KL | −0.657969 |
| ft_:_KMT2D_\|_variant_type_−_equals_−_SV_+_isKL_−_equals_−_KL | −0.0542613 |
| ft_:_BRCA1_\|_isKL_−_equals_−_KL | −0.2540207 |
| ft_:_DUSP2_\|_variant_type_−_equals_−_SV_+_codon_−_equals_−_134 | −0.8152089 |
| ft_:_PIM1_\|_variant_type_−_equals_−_SV_+_codon_−_equals_−_135 | −0.1497665 |
| ft_:_PTPN11_\|_isKL_−_equals_−_KL | −0.1167284 |
| ft_:_BTG2_\|_variant_type_−_equals_−_SV_+_codon_−_equals_−_48 | −0.0152634 |
| ft_:_BTG2_\|_variant_type_−_equals_−_SV_+_codon_−_equals_−_45 | 0.02565534 |
| ft_:_CCND3_\|_variant_type_−_equals_−_SV_+_codon_−_equals_−_190 | 0.57357736 |
| ft_:_DAXX_\|_variant_type_−_equals_−_SV_+_codon_−_equals_−_455 | −0.3863176 |
| ft_:_FBXO11_\|_isKL_−_equals_−_KL | 0.12557906 |
| ft_:_MAPK1_\|_variant_type_−_equals_−_SV_+_codon_−_equals_−_1 | 0.20638698 |
| ft_:_IGH_\|_variant_type_−_equals_−_RE_+_gene2_−_equals_−_BCL2_+_isKL_− | 0.42225842 |
| ft_:_KDM6A_\|_variant_type_−_equals_−_SV_+_isKL_−_equals_−_KL | −0.382399 |
| hotspots_STAT6_417_419 | 0.42139716 |
| ft_:_BCL6_\|_variant_type_−_equals_−_RE_+_gene2_−_equals_−_IGH_+_isKL_− | −0.1259457 |
| ft_:_B2M_\|_variant_type_−_equals_−_SV_+_codon_−_equals_−_1 | 0.74380662 |
| ft_:_PTPN6_\|_variant_type_−_equals_−_SV_+_isKL_−_equals_−_KL | −0.1007704 |
| ft_:_TP53_\|_variant_type_−_equals_−_SV_+_codon_−_equals_−_273 | 0.00758395 |
| ft_:_KMT2D_LisKL_−_equals_−_KL | −0.0167986 |
| ft_:_HIST1H1E_\|_variant_type_−_equals_−_SV_+_isKL_−_equals_−_KL | 0.18457867 |
| ft_:_CARD11_\|_variant_type_−_equals_−_SV_+_codon_−_equals_−_230 | −0.0008312 |
| CDKN2A_or_B_del | −0.6592701 |
| ft_:_PTPN11_\|_variant_type_−_equals_−_SV_+_codon_−_equals_−_197 | −0.0001514 |
| ft_:_PIM1_\|_variant_type_−_equals_−_SV_+_codon_−_equals_−_97 | −0.8007032 |
| ft_:_CD79B_\|_variant_type_−_equals_−_SV_+_codon_−_equals_−_196 | −0.0960342 |
| ft_:_CDKN2B_\|_variant_type_−_equals_−_RE_+_isKL_−_equals_−_KL | −0.7328949 |
| ft_:_MYC_\|_variant_type_−_equals_−_RE_+_gene2_−_equals_−_IGH_+_ | −0.0427746 |
| isKL_−_equals_− | |
| ft_:_MYD88_\|_variant_type_−_equa1s_−_SV_+_codon_−_equa1s_−_265 | −0.359751 |
| ft_:_DDX3X_\|_variant_type_−_equals_−_SV_+_isKL_−_equals_−_KL | 0.06998015 |
| ft_:_PIM1_\|_variant_type_−_equa1s_−_SV_+_codon_−_equa1s_−_30 | −0.0375632 |
| ft_:_BCL10_1_isKL_−_equals_−_KL | −0.1240489 |
| ft_:_TBL1XR1_\|_variant_type_−_equals_−_SV_+_isKL_−_equals_−_KL | −0.4020574 |
| hotspots_KLHL6_547_568_65_90 | 0.0732899 |
| ft_:_IGH_\|_variant_type_−_equals_−_RE_+_gene2_−_equals_−_BCL6_+_isKL_− | −0.0026765 |
| ft_:_IRF4_\|_variant_type_−_equals_−_SV_+_codon_−_equals_−_70 | −0.2206269 |
| ft_:_PRDM1_\|_isKL_−_equals_−_KL | −0.3567579 |
| ft_:_CCND3_\|_variant_type_−_equals_−_SV_+_isKL_−_equals_−_KL | −0.0157609 |
| ft_:_BCL2_\|_variant_type_−_equals_−_RE_+_isKL_−_equals_−_KL | 0.02093477 |
| ft_:_HIST1H2AM_\|_variant_type_−_equals_−_SV_+_isKL_−_equals_−_KL | −0.0517756 |
| ft_:_KLHL6_\|_variant_type_−_equals_−_SV_+_codon_−_equals_−_568 | 0.03590108 |
| ft_:_KDM6A_\|_isKL_−_equals_−_KL | −0.0095082 |
| ft_:_PTPN6_\|_isKL_−_equals_−_KL | −0.0194835 |
| ft_:_PTPN11_\|_variant_type_−_equals_−_SV_+_isKL_−_equals_−_KL | −0.0014204 |
| ft_:_ETS1_\|_variant_type_−_equals_−_SV_+_codon_−_equals_−_17 | −0.2238722 |
| ft_:_PIM1_\| _variant_type_−_equals_−_SV_+_codon_−_equals_−_10 | −0.0111813 |

### Example 4: Statistics

Feature enrichment was assessed using the Nanostring assigned COO. Binary feature enrichment was determined using a Fisher exact test. Continuous feature enrichment was determined using a Mann-Whitney-Wilcox test. Univariate Hazard ratios and p-values for the association of COO subtypes with PFS were calculated using Cox regression.

### Example 5: Mutational Signatures

Mutational signatures were determined as described by Zehir et al. (Zehir, A., et al. Mutational landscape of metastatic cancer revealed from prospective clinical sequencing of 10,000 patients. Nat Med 23, 703-713 (2017)). Briefly, trinucleotide matrices were decomposed into the 30 COSMIC signatures (Alexandrov, L. B., et al. Signatures of mutational processes in human cancer. Nature 500, 415-421 (2013)). Signatures were aggregated to APOBEC (signatures 2 and 13); smoking (signature 4), BRCA (signature 3); MMR (signatures 1, 6, 15, 20 and 26); UV (signature 7); POLE (signature 10); and Alkylating (signature 11). All point mutations were included in the analysis with the exception of driver alterations and predicted germline alterations. A sample was deemed to have a dominant signature if a mutational class harbored a score of 0.4 or greater. For the genome-wide calculation, mutations were considered in all regions baited for short variant detection excluding regions with known sequencing artifacts. Samples were only considered if they had 20 or more assessable mutations. For the IGH analysis, mutations were assessed in selected regions of the genomic IGH locus (chr14:106211312-106382700 hg19).

### Example 6: COO DNA Classifier: A Genomically Defined Model for Cell of Origin

482 DLBCL samples from the GOYA study that had DNA sequenced using FoundationOne®Heme were split into two sets: ⅔ into a training set and ⅓ into a held-out validation set. The training set was further subset to include only samples with either an ABC or GCB call as determined by Nanostring; unclassified samples or samples without a call were excluded. The held-out validation set was used after the model was complete to determine concordance. The training set data were then used to train a penalized logistic regression model to identify ABC or GCB samples using DNA-based features without the need for RNA. 594 features were available to train the model, including binary features of any alteration in a gene, specific alterations (codons), and hotspot alterations (any one of multiple codons) that occurred at least 5 times in the GOYA dataset as well as derived DNA-based features such as tumor mutational burden (TMB), chromosome arm-level copy number and zygosity metrics, and frequency of alteration classes (e.g., T mutated to A). Per-sample probabilities were extracted from the model, and a pair of cutoffs was chosen to optimize sensitivity and specificity, with particular focus on optimizing ABC accuracy. This model is herein referred to as the Cell of Origin DNA Classifier (COODC; the classifier is also referred to herein, e.g., as a method, model and assay). The COODC model contained a total of 74 genomic features and generated a continuous probability score of a sample being ABC ranging from 0 to 0.999. The 74 genomic features included 18 arm-level alteration features, including copy number and loss of heterozygosity features, 32 gene short variant features, 6 rearrangement-based features, 13 gene-level features (including copy number, rearrangement, and short variant alterations) and various other summary features (including T>A mutation prevalence).

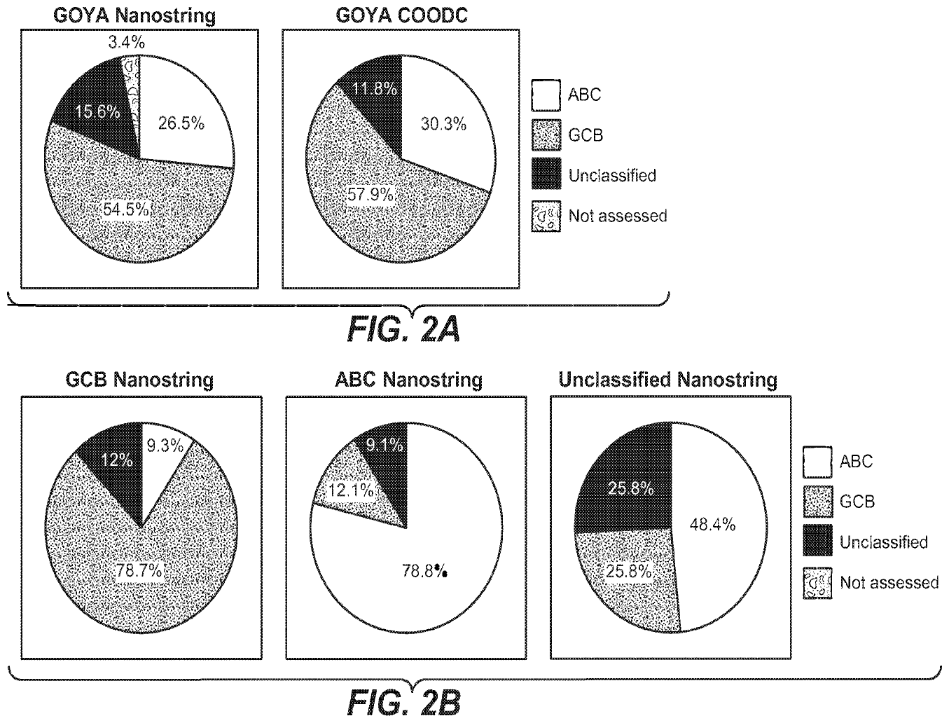

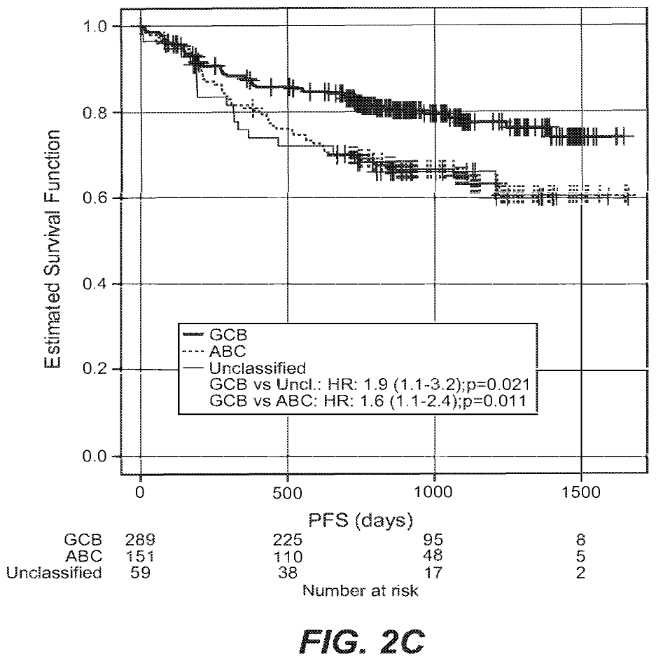

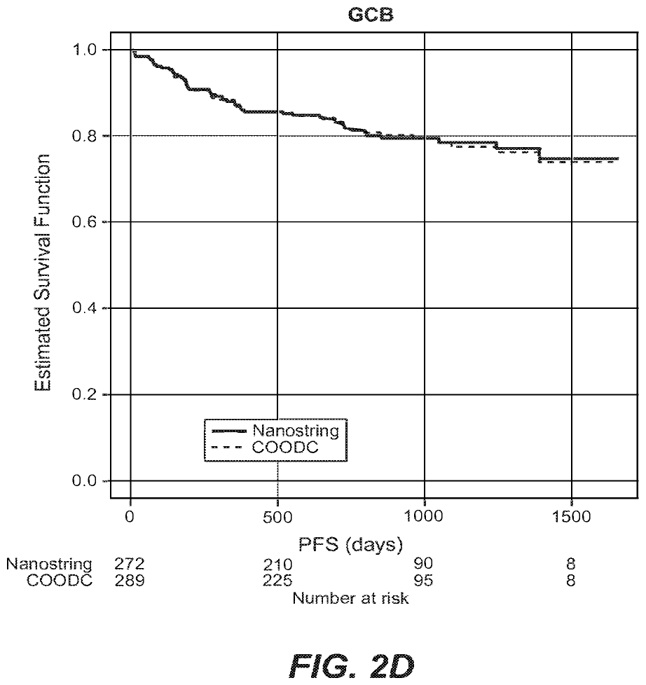

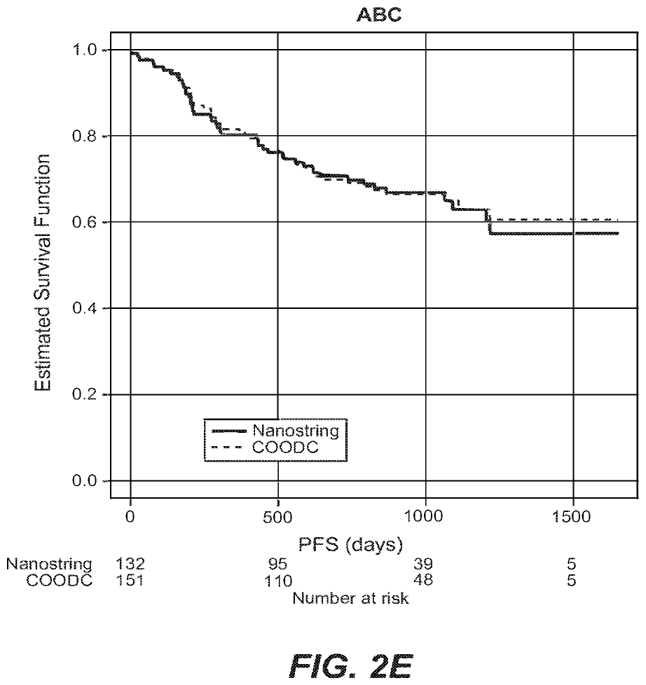

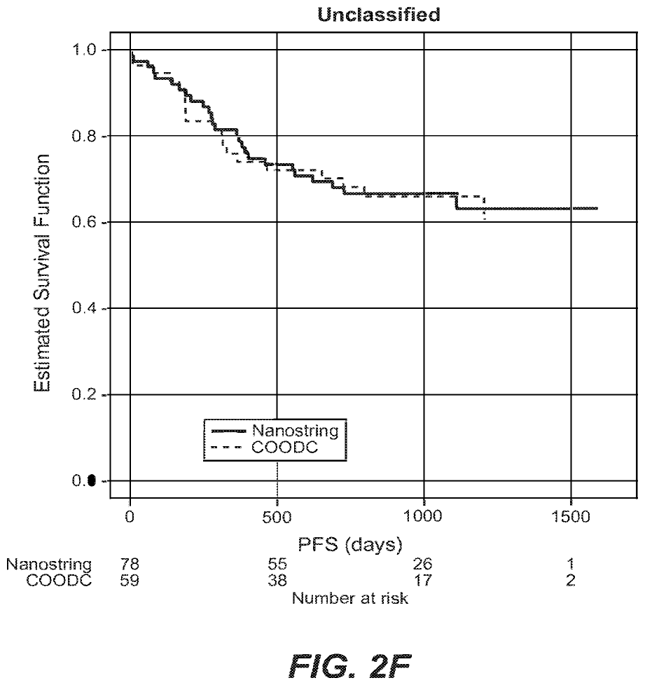

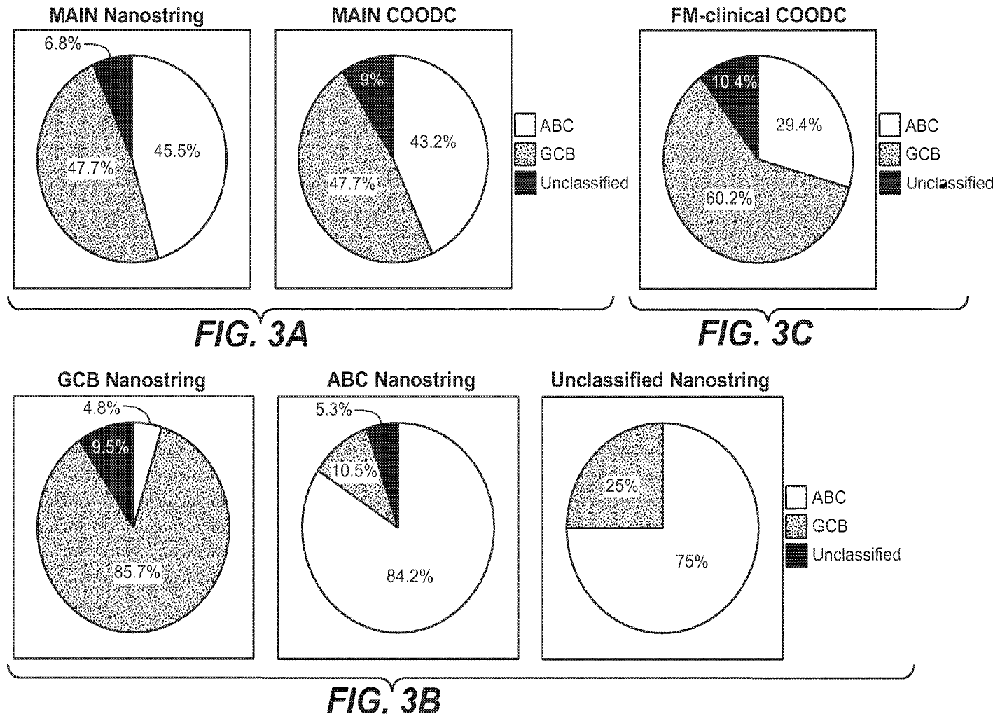

To define optimal cutoffs for the continuous probability score, the “best” cutoff from the ROC model (FIGS. 1A and 1B) was determined, i.e. the point with the highest total sensitivity and specificity. A 0.1 probability buffer was then added on either side of the optimal cut-point in order to define an unclassified region. This resulted in similar subtype breakdowns across the entire GOYA dataset, with 57.9% GCB, 30.3% ABC, and 11.8% unclassified as determined by the COODC compared to 54.5% GCB, 26.5% ABC, and 15.6% unclassified (3.4% of cases were not submitted for Nanostring COO typing), as determined by Nanostring (FIG. 2A). This results in 85 (88.5%) concordant calls within the held-out validation set when considering 96 samples with either an ABC or GCB call from both the COODC and Nanostring methods (unclassified samples by either method were excluded) (Table 2). For this assessment, all unclassified calls were excluded. However, in keeping with the assessment provided by Scott et al. (Scott, D. W., et al. Determining cell-of-origin subtypes of diffuse large B Cell lymphoma using gene expression in formalin-fixed paraffin-embedded tissue. Blood 123, 1214-1217 (2014)), where only unclassified from the ‘gold standard’ were excluded, it was found 90% (97/108) concordance with Nanostring (Table 2). Of 75 samples Nanostring considered GCB, 59 (78.7%) were also called GCB by COODC, 9 (12%) were called unclassified, and 7 (9.3%) were incorrectly called ABC (FIG. 2B and Table 2). Similarly, of 33 ABC calls by Nanostring, 78.8% (26) were called ABC by COODC, 9.1% (3) were called unclassified, and 12.1% (4) were incorrectly called GCB (FIG. 2B and Table 2). In addition, the concordance with Nanostring was assessed on an independent study cohort (MAIN study) of 44 samples. In this cohort, COODC demonstrated continued high concordance with 91.9% accuracy (FIGS. 3A, 3B, and 3C and Table 3). The COODC model was further applied to an independent set of 597 FM-clinical samples (FIGS. 3A, 3B, and 3C). Although there is no gold standard COO assessment to compare with, similar breakdowns of COO type by COODC were found as in the GOYA samples, with 60% GCB, 30% ABC, and 10% unclassified.

TABLE 2
Held-out Validation Set

| | | Nanostring | | |
|---|---|---|---|---|
| Held-out Validation set | | ABC | GCB | UNCLASSIFIED |
| COODC | ABC | 26 | 7 | 15 |
| | GCB | 4 | 59 | 8 |
| | UNCLASSIFIED | 3 | 9 | 8 |

TABLE 3
Held-out Validation Set

| | | Nanostring | | |
|---|---|---|---|---|
| | | ABC | GCB | UNCLASSIFIED |
| COODC | ABC | 16 | 1 | 3 |
| | GCB | 2 | 18 | 1 |
| | UNCLASSIFIED | 1 | 2 | 0 |

Overall, progression free survival (PFS) estimates for the COODC classifications were highly consistent with the Nanostring COO calls (FIG. 2D), and it was observed to significantly reduced PFS among samples classified as ABC (HR: 1.6; 95% confidence interval (CI) [1.1-2.4]; p=0.011) or unclassified (HR: 1.9; 95% CI [1.1-3.2]; p=0.021) when compared to GCB samples. This indicates that stratifying patient prognosis can be achieved utilizing a DNA only COO model such as COODC.

### Example 7: Biological Significance of Features Included in the COODC Model

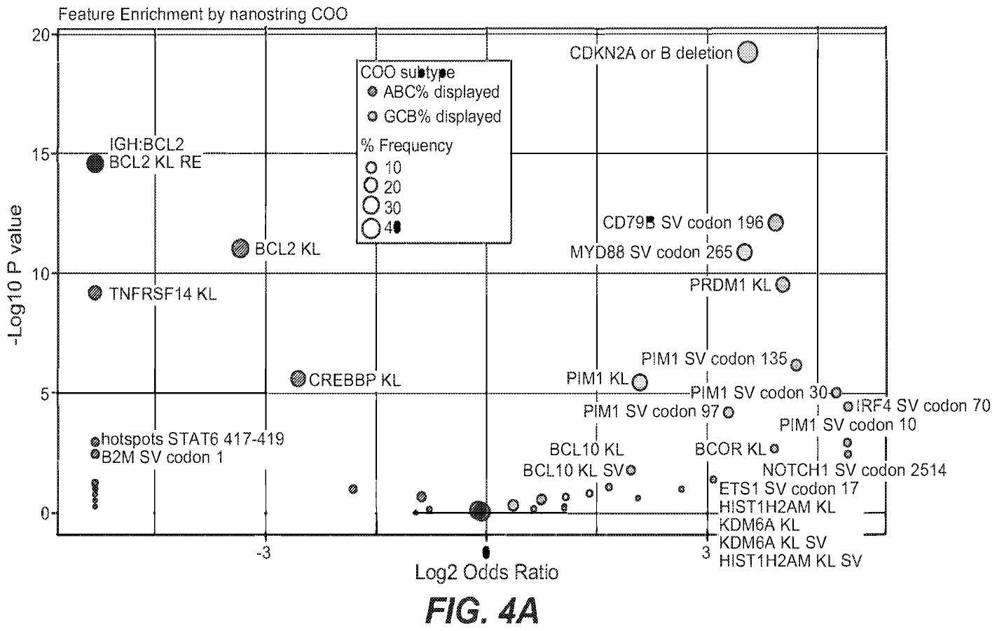

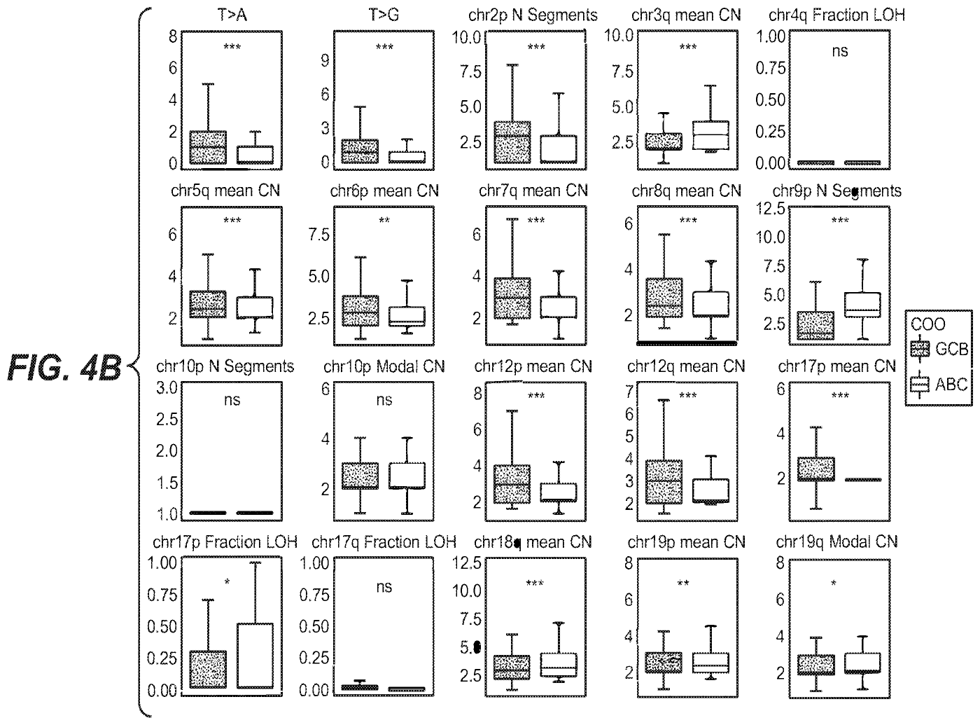

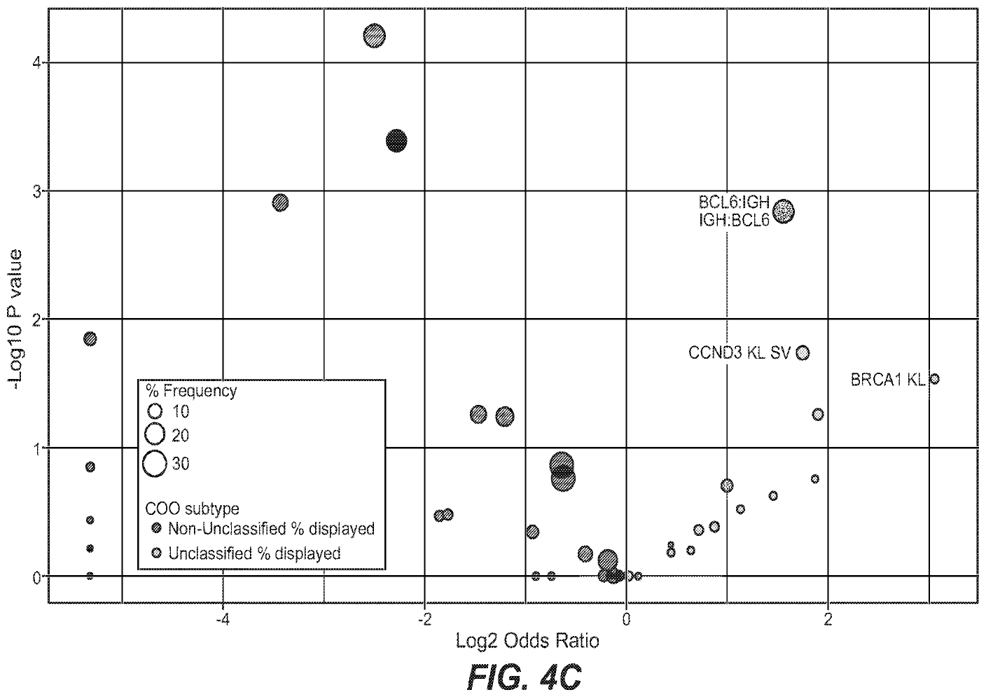

Because an unbiased approach was taken to feature selection, the potential biological significance of the features included in the COODC model was of interest. It was found that a number of expected features, including IGH:BCL2 rearrangements, CREBBP alterations, and TNFRSF14 alterations are all highly enriched in GCB samples. Chromosome 3q copy number increase, CDKN2A/B deletions, CD79B alterations at amino acid 196, and MYD88 alterations at amino acid 265 were highly enriched in ABC samples (FIG. 4A). A few new features of particular interest, including the frequency of T>A and T>G alterations, the average copy number of chromosome 6p and the number of copy number segments of chromosome 9p (FIG. 4B) were also identified. Both T>A and T>G alteration frequencies in non-driver alterations is higher in GCB (p<0.0001 for both) (FIG. 4B). A lower mean copy number of chromosome 6p, which encodes the HLA locus, was found in ABC (p=0.0064). The number of modeled segments on chromosome 9p, including CD274 and PDCD1LG2, is higher in ABC (p<0.0001) suggesting that regions of this arm are fractured, leading to differences in copy number. These two components, which include the number of modeled segments on chromosome 9p and the lower copy number of chromosome 6p, point to potential biological significance for immune evasion.

In addition to features specifically enriched in ABC or GCB, there was evidence of the truly intermediate nature of unclassified samples. Enrichment of novel features was identified, such as IGH:BCL6 rearrangements (FIG. 4C), as well as enrichment of ABC or GCB-like features, such as the average copy number of chromosome 6p in which unclassified is similar to GCB, and the T>A alteration frequency in which unclassified is similar to ABC (FIG. 4B). Many of the features included in the model were not significantly enriched in ABC or GCB subtypes. This suggests that some features may be important aspects of prediction only in combination with or in the absence of other features.

### Example 8: Mutational Signatures

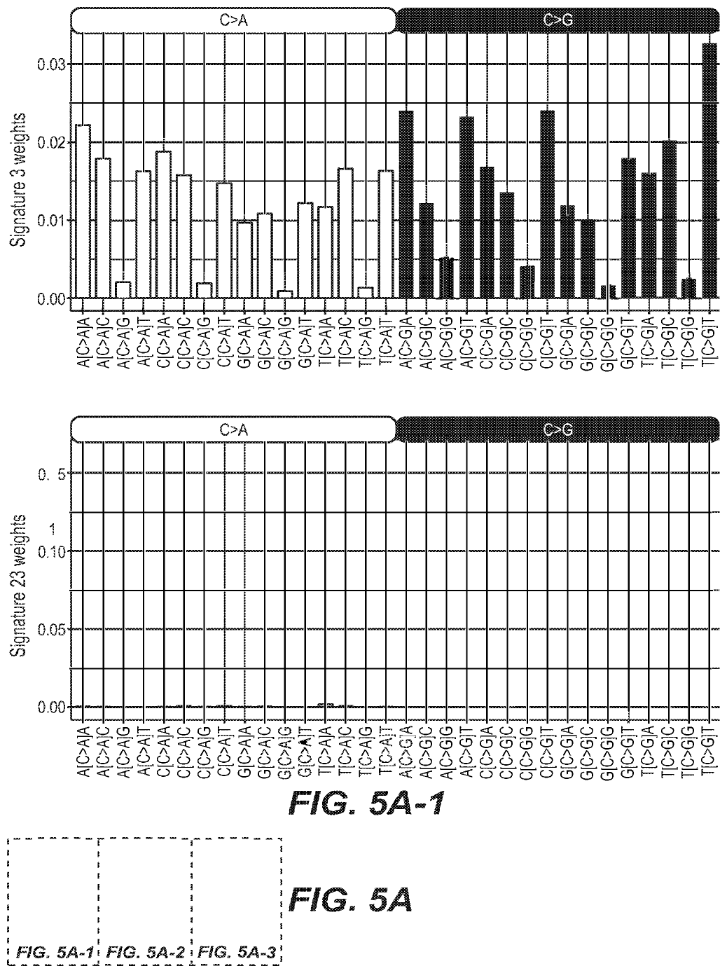

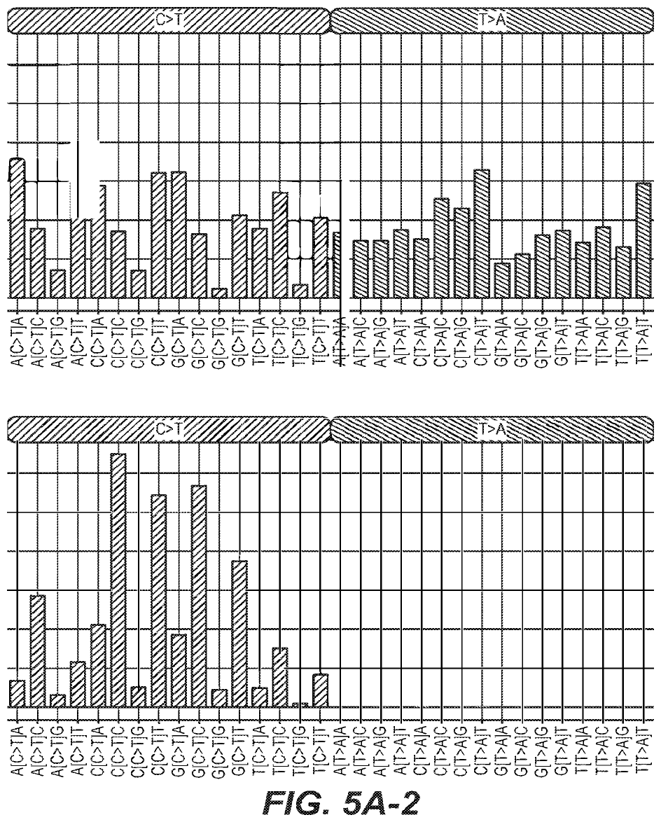

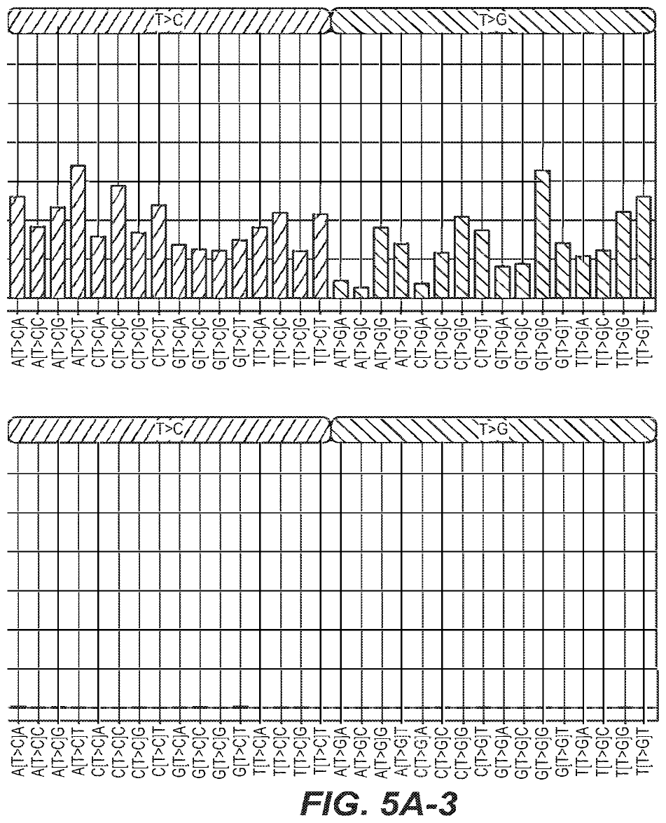

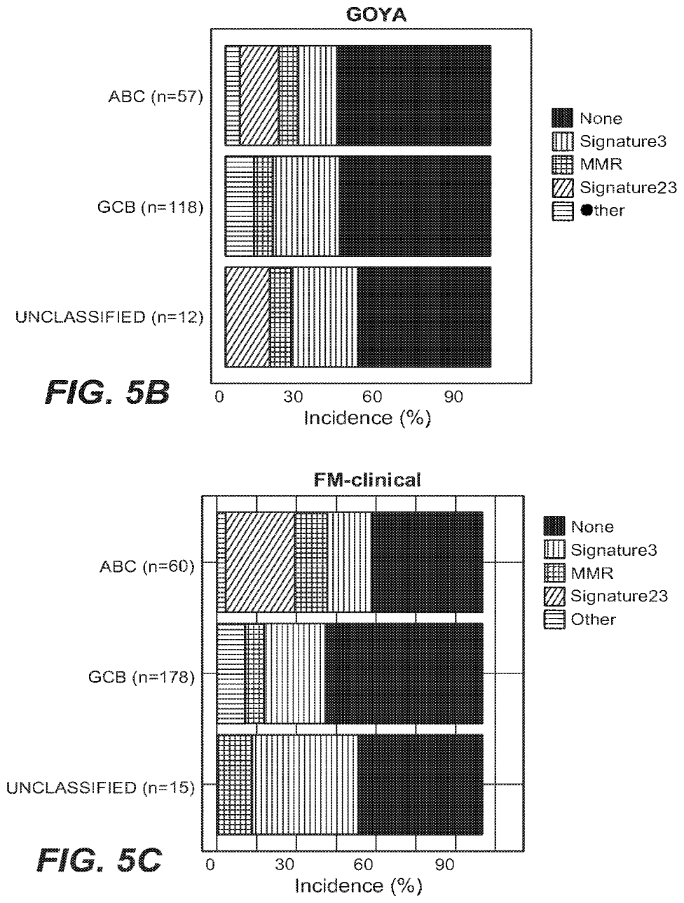

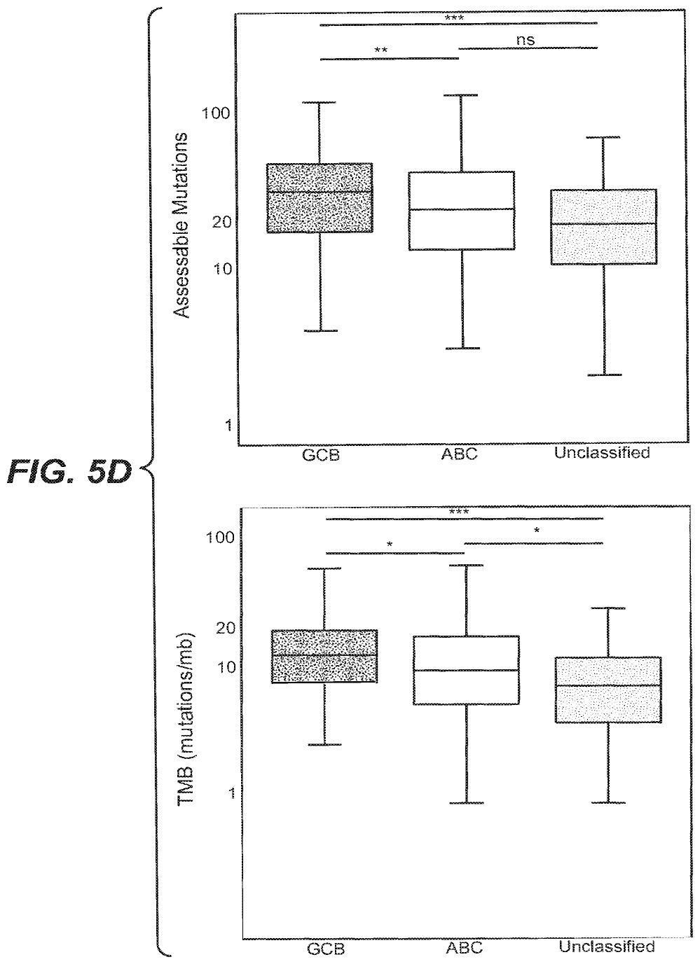

The observed difference in T>A and T>G alterations resulted in an investigation of potential differences in mutational signatures. Given the known role of AID in SHM and CSR in normal germinal center B Cells, the proposed role in transformation to lymphoma, and the recent data from Chapuy showing distinct mutational signatures in DLBCL, it was investigated whether there was evidence of the AID mutational processes across all the DNA alterations. Using trinucleotide mutational signatures identified in COSMIC, it was found that 26.3% of COODC-GCB samples compared to 15.8% of ABC samples (p=0.13) have a dominant COSMIC signature 3, while 14% of ABC samples compared to 1.7% of GCB samples (p=0.002) have a dominant signature 23 (FIG. 5B).

Signature 23 is dominated by the trinucleotide context in which C is altered to T (FIG. 5A). Of the main trinucleotide contexts, ⅗ are found in the R[C]Y context (FIG. 5A). This signature is similar to WR[C]Y, a common context for AID targeting somatic hypermutation, suggesting a strong AID mutational signature in ABC. Notably, this was not the result of fewer mutations overall in the GCB subset; the total mutation count was higher on average in GCB subsets compared with ABC (31 in GCB vs 23.5 in ABC, p=0.002) (FIG. 5D). Interestingly, the predominant COSMIC signature in GCB samples is signature 3, which has been annotated as a “BRCA” signature. Given the similarities between resolution of DSB in the absence of BRCA, which utilizes non-homologous end joining (NHEJ) and the resolution of DSB induced by AID as part of CSR, which also primarily utilizes NHEJ, it is presumed that signature 3 may identify the mutational scars of DSB repair, and in this context, may represent an AID-DSB signature.

### Example 9: Other Genetically-Defined Subsets of DLBCL

In light of the recent publications highlighting the use of genetically defined DLBCL subsets to stratify further good and poor prognosis groups, subsets in data from this targeted NGS panel were investigated. Given the similarities between the subsets identified by Schmitz et al. and Chapuy et al. (MCD is similar to C5, EZB is similar to C3, and BN2 is similar to C1 as classified by Schmitz and Chapuy respectively), approximate definitions corresponding to the “seed” for these specific subsets, including CD79B alterations or MYD88 L265P for MCD/C5, BCL6 rearrangement or NOTCH2 alteration for BN2/C1, EZH2 alteration or BCL2 rearrangement for EZB/C3, and NOTCH1 alteration for N1 were identified.

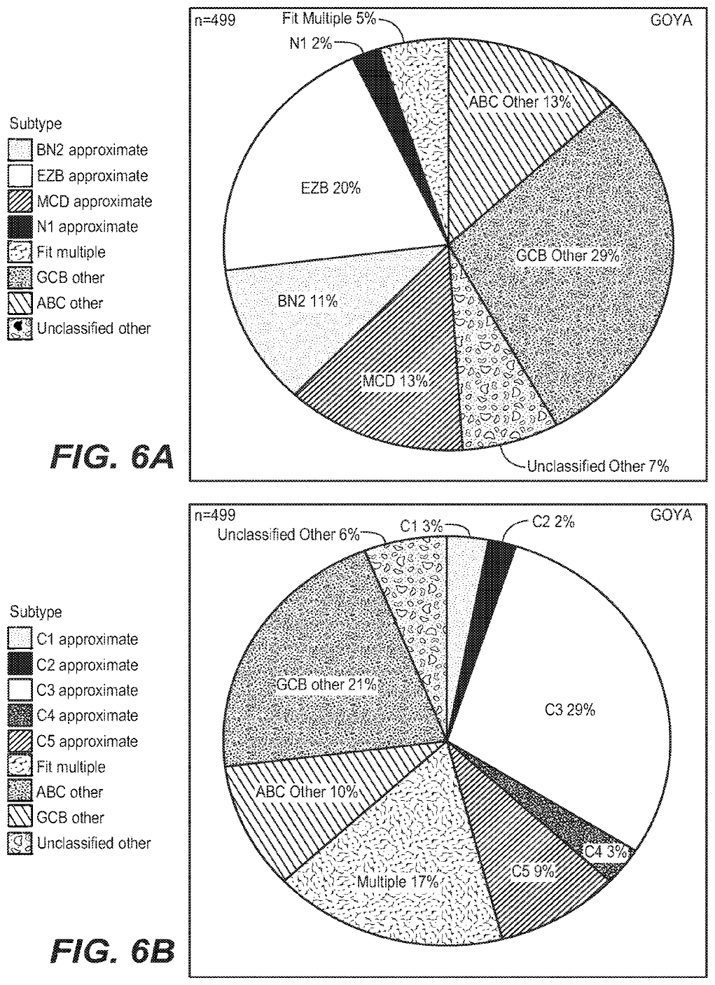

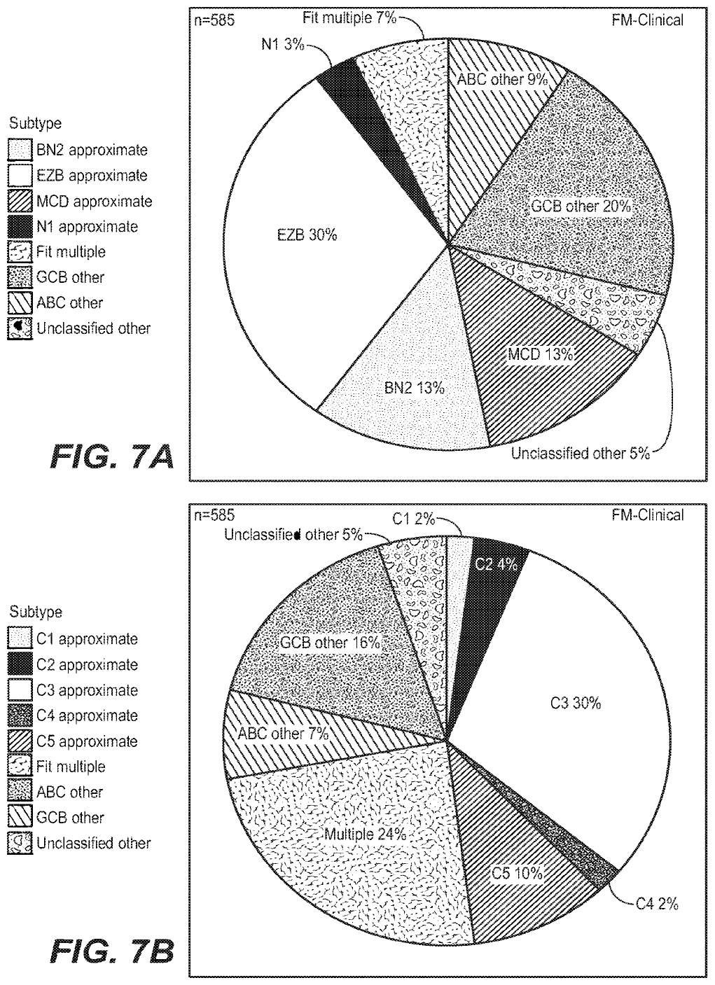

Using these simplified genetically defined groups, 46% of GOYA and 57% of FM-clinical samples could be classified into one of these groups, while 49% of GOYA and 37% of FM-clinical samples could not be, and the remaining 5% of GOYA and 6% of FM-clinical samples could be classified into more than one of these genetically defined groups (FIGS. 6A and 6B, FIGS. 7A and 7B).

In addition to these groups that are similar to those recently defined as potentially of prognostic value by Schmitz and colleagues and Chapuy and colleagues, subgroups with therapeutic value (Table 4 and Table 5) were identified using the COODC disclosed herein. It was possible to identify BCR-independent and BCR-dependent subgroups using CD79B alterations with ABC, with predictive implications for ibrutinib as shown by Wilson and colleagues (Wilson, W. H., et al. Targeting B cell receptor signaling with ibrutinib in diffuse large B cell lymphoma. Nature Medicine 21, 922 (2015)). It was also found that a BCR-dependent subgroup, defined by the presence of a CD79B predicted deleterious alteration, are present in 24.5% of the ABC GOYA study samples (9.4% overall in GOYA) and 21.9% of the ABC FM-clinical samples (7.9% overall in FM-clinical samples). It was additionally observed that 79.2% of GOYA samples and 63.8% of FM-clinical samples were TP53 wild type, suggesting a potential relevance for MDM2 inhibitors in these cohorts. EZH2 inhibitors may be clinically relevant in the 10% of GOYA samples and 12.7% of FM-clinical samples with EZH2 alterations, while BCL2 inhibitors could be considered in the 23.2% of GOYA samples and 32.5% of FM-clinical samples with IGH:BCL2 rearrangements, BCL2 amplifications, or BCL2 short variants (Table 4 and Table 5). Overall, this suggests that up to 85% of the DLBCL samples in the tested cohorts could be eligible for targeted therapy using an MDM2 inhibitor, an EZH2 inhibitor, a BCL2 inhibitor, or a BCR pathway inhibitor.

TABLE 4
Additional molecularly defined subgroups for GOYA

| COODC Subtype | ALL | GCB | ABC | UNCLASSIFIED |
|---|---|---|---|---|
| Schmitz MCD-like | 12.4 | 2.1 | 34.4 | 6.8 |
| Schmitz BN2-like | 14.4 | 13.8 | 12.6 | 22.0 |
| Schmitz EZB-like | 19.2 | 31.1 | 0.7 | 8.5 |
| Schmitz N1-like | 2.4 | 1.0 | 4.0 | 5.1 |
| Multiple_Schmitz | 6.2 | 5.5 | 7.9 | 5.1 |
| Chapuy C1-like | 3.2 | 3.8 | 2.0 | 3.4 |
| Chapuy C2-like | 1.8 | 1.0 | 2.6 | 3.4 |
| Chapuy C3-like | 29.3 | 40.8 | 9.9 | 22.0 |
| Chapuy C4-like | 2.6 | 3.1 | 1.3 | 3.4 |
| Chapuy C5-like | 9.0 | 2.1 | 22.5 | 8.5 |
| Multiple Chapuy | 17.0 | 12.5 | 27.8 | 11.9 |
| BCR-dependent (CD79B+) | 9.4 | 1.7 | 24.5 | 8.5 |
| Double_Hit | 1.4 | 2.1 | 0.0 | 1.7 |
| TP53_WT | 78.8 | 77.5 | 86.1 | 66.1 |
| BCL2 alteration | 23.2 | 33.6 | 9.9 | 6.8 |
| EZH2 alteration | 10.0 | 15.9 | 1.3 | 3.4 |
| Targetable | 85.0 | 86.2 | 88.7 | 69.5 |
| B2M alteration | 15.8 | 19.0 | 11.3 | 11.9 |
| CD274 amplification | 4.4 | 4.8 | 4.0 | 3.4 |
| CD58 alteration | 8.2 | 8.3 | 9.9 | 3.4 |
| CD70 alteration | 8.8 | 8.3 | 9.3 | 10.2 |
| CIITA alteration | 4.8 | 6.2 | 2.6 | 3.4 |
| Immune Evasion | 33.9 | 35.6 | 32.5 | 28.8 |

TABLE 5
COO plus FM-Clinical

| COODC Subtype | ALL | GCB | ABC | UNCLASSIFIED |
|---|---|---|---|---|
| Schmitz MCD-like | 13.0 | 1.1 | 38.4 | 9.8 |
| Schmitz BN2-like | 12.8 | 9.9 | 13.4 | 27.9 |
| Schmitz EZB-like | 29.7 | 47.4 | 2.3 | 4.9 |
| Schmitz N1-like | 2.6 | 0.9 | 5.8 | 3.3 |
| Multiple_Schmitz | 7.0 | 7.1 | 8.1 | 3.3 |
| Chapuy C1-like | 2.2 | 1.7 | 2.3 | 4.9 |
| Chapuy C2-like | 4.1 | 2.6 | 7.0 | 4.9 |
| Chapuy C3-like | 29.9 | 42.9 | 9.9 | 11.5 |
| Chapuy C4-like | 1.9 | 1.4 | 2.9 | 1.6 |
| Chapuy C5-like | 10.3 | 2.8 | 26.7 | 6.6 |
| Multiple Chapuy | 23.8 | 22.7 | 27.3 | 19.7 |
| BCR-dependent (CD79B+) | 8.0 | 1.4 | 22.7 | 4.9 |
| Double_Hit | 1.7 | 2.8 | 0.0 | 0.0 |
| TP53_WT | 62.7 | 61.1 | 68.0 | 57.4 |
| BCL2 alteration | 32.8 | 50.3 | 5.8 | 8.2 |
| EZH2 alteration | 13.0 | 20.2 | 2.3 | 1.6 |
| Targetable | 80.7 | 86.4 | 75.0 | 63.9 |
| B2M alteration | 15.9 | 21.0 | 7.0 | 11.5 |
| CD274 amplification | 2.7 | 2.0 | 4.7 | 1.6 |
| CD58 alteration | 9.4 | 8.2 | 13.4 | 4.9 |
| CD70 alteration | 6.8 | 8.0 | 6.4 | 1.6 |
| CITTA alteration | 5.3 | 6.8 | 3.5 | 1.6 |
| Immune Evasion | 31.1 | 34.1 | 29.1 | 19.7 |

The Examples are offered for illustrative purposes only, and are not intended to limit the scope of the present invention in any way. Indeed, various modifications in addition to those shown and described herein will become apparent to those skilled in the art from the foregoing description and fall within the scope of the appended claims.

1-73. (canceled)

74. A method of determining the cell of origin (COO) of a diffuse large B cell lymphoma (DLBCL) of a patient and treating the patient, the method comprising:
extracting DNA from a sample from the patient,
amplifying the extracted DNA,
sequencing the amplified DNA to acquire a list of genomic features associated with the sample,
applying, by a computer, a COO DNA classification (COODC) model to the list of genomic features to calculate a probability score,
based on the probability score:
classifying, by the computer, the patient as having a germinal center B Cell (GCB) COO when the probability score is below a first pre-defined cutoff, and
classifying, by the computer, the patient as having an activated B cell (ABC) COO when the probability score is above or equal to a second pre-defined cutoff, and
based on the patient classification, administering a therapy.

75. The method of claim 74, wherein the first pre-defined cutoff is between 0.2-0.3, and the second pre-defined cutoff is between 0.4-0.6.

76. The method of claim 74, wherein no RNA analysis is performed.

77. The method of claim 74, wherein RNA analysis does not contribute to the patient classification.

78. The method of claim 74, wherein no immunohistochemistry (IHC) analysis is performed.

79. The method of claim 74, wherein IHC analysis does not contribute to the patient classification.

80. The method of claim 74, wherein the therapy is a therapy that is effective for treating GCB COO.

81. The method of claim 74, wherein the therapy is a therapy that is effective for treating ABC COO.

82. The method of claim 74, wherein the sample is a clinical sample.

83. The method of claim 82, wherein the clinical sample is selected from the group consisting of a tumor biopsy, blood, bone marrow aspirate, cell-free DNA, circulating tumor DNA, and circulating tumor cells.

84. The method of claim 83, wherein the clinical sample is cell-free DNA.

85. The method of claim 83, wherein the tumor biopsy is preserved as a formaldehyde or paraformaldehyde-fixed paraffin-embedded (FFPE) tissue preparation.
